(19) United States
(12) Patent Application Publication
(10) Pub. No.: US 2025/0195096 A1
(43) Pub. Date: Jun. 19, 2025

(54) SURGICAL INSTRUMENT INCLUDING ELECTRICAL AND FLUID ISOLATION FEATURES

(71) Applicant: Intuitive Surgical Operations, Inc., Sunnyvale, CA (US)

(72) Inventors: Matthew A. WIXEY, Rochester, CT (US); Erik NELSON, Durango, CO (US); Gal ZEIDMAN, Mountain View, CA (US)

(73) Assignee: Intuitive Surgical Operations, Inc., Sunnyvale, CA (US)

(21) Appl. No.: 18/846,477

(22) PCT Filed: Mar. 7, 2023
(86) PCT No.: PCT/US2023/014700

§ 371 Date: Sep. 12, 2024

Related U.S. Application Data
(60) Provisional application No. 63/319,926, filed on Mar. 15, 2022.

(51) Int. Cl. A61B 17/29; A61B 34/00; A61B 34/35

(52) U.S. Cl. CPC A61B 17/29; A61B 34/35; A61B 34/71; A61B 2017/2948

(57) ABSTRACT

A medical device includes an instrument shaft, a tension element guide, a tension element, and a semisolid sealing substance. The instrument shaft includes a proximal end portion, a distal end portion, and a channel between the proximal end portion and the distal end portion. The tension element guide is at the distal end of the instrument shaft, and includes a proximal seal, a distal seal, and a middle seal between the proximal seal and the distal seal. A sealing substance volume is defined between the proximal seal and the middle seal and holds the sealing substance. The tension element extends from the channel of the instrument shaft, past the proximal seal, through the sealing substance in the sealing substance volume, past the middle seal, past the distal seal, and beyond the tension element guide.

### CROSS-REFERENCE TO RELATED APPLICATIONS

This application claims benefit of priority to U.S. Provisional Application Ser. No. 63/319,926, entitled “Surgical Instrument Including Electrical and Fluid Isolation Features,” filed Mar. 15, 2022, which is incorporated herein by reference in its entirety.

### BACKGROUND

The embodiments described herein relate to medical devices, and more specifically to endoscopic tools. More particularly, the embodiments described herein relate to medical devices that include mechanisms for providing fluid and electrical isolation within the medical devices.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft are inserted into a small incision or a natural orifice of a patient to position the end effector at a work site within the patient's body. The optional wrist mechanism can be used to change the end effector's orientation with reference to the shaft to perform the desired procedure at the work site. Known wrist mechanisms generally provide the desired mechanical degrees of freedom (DOFs) for movement of the end effector. For example, known wrist mechanisms are able to change the pitch and yaw orientation of the end effector with reference to the shaft's longitudinal axis. A wrist may optionally provide a roll DOF for the end effector with reference to the shaft, or an end effector roll DOF may be implemented by rolling the shaft, wrist, and end effector together as a unit. An end effector may optionally have additional mechanical DOFs, such as grip or knife blade motion. In some instances, wrist and end effector mechanical DOFs may be combined to provide various end effector control DOFs. For example, U.S. Pat. No. 5,792,135 (filed May 16, 1997) discloses a mechanism in which wrist and end effector grip mechanical DOFs are combined to provide an end effector yaw control DOF.

To enable the desired movement of the distal wrist mechanism and end effector, known instruments include cables that extend through the shaft of the instrument and that connect the wrist mechanism to a mechanical structure configured to move the cables to operate the wrist mechanism and end effector. For teleoperated systems, the mechanical structure is typically motor driven and is operably coupled to a computer processing system to provide a user interface for a clinical user (e.g., a surgeon) to control the instrument as a whole, as well as the instrument's components and functions.

Patients benefit from continual efforts to improve the effectiveness of MIS methods and devices. For example, reducing the size and/or the operating footprint of the shaft and wrist mechanism can allow for smaller entry incisions and reduced need for space at the surgical site, thereby reducing the negative effects of surgery, such as pain, scarring, and undesirable healing time. But producing small medical devices that implement the clinically desired functions for minimally invasive procedures can be challenging. Specifically, simply reducing the size of known wrist mechanisms by scaling down the components will not result in an effective solution because required component and material properties do not scale at relatively small physical dimensions. For example, efficient implementation of a wrist mechanism can be complicated because the cables must be carefully routed through the wrist mechanism to maintain cable tension throughout the range of motion of the wrist mechanism or end effector and to minimize the interactions (coupling effects) of motion about one rotation axis upon motion about another rotation axis. As another example, pulleys and/or contoured surfaces are generally needed to reduce cable friction, which extends instrument life and permits operation without excessive forces being applied to the cables or other structures in the wrist mechanism. But increased localized forces that may result from smaller structures and cable bend radii (including smaller diameter cables and other wrist and end effector components) can result in undesirable lengthening (e.g., stretch or creep) of the cables during storage and use, reduced cable life, and the like.

Further, the wrist mechanism generally provides specific degrees of freedom for movement of the end effector. For example, for forceps or other grasping tools, the wrist may be able to change the end effector pitch, yaw, and grip orientations with reference to the instrument shaft. More degrees of freedom could be implemented through the wrist but would require additional actuation members (e.g., cables) in the wrist and shaft, and these additional members compete for the limited space that exists given the size restrictions required by MIS applications. Components needed to actuate other degrees of freedom, such as end effector roll or insertion/withdrawal through movement of the main tube, also compete for space at or in the shaft of the device.

A conventional architecture for a wrist mechanism in a manipulator-driven medical device uses cables pulled in and payed out by a capstan in the proximal mechanical structure and thereby rotate the portion of the wrist mechanism that is connected to the capstan via the cables. For example, a wrist mechanism can be operably coupled to three capstans-one each for rotations about a pitch axis, a yaw axis, and a grip axis. Each capstan can be controlled by using two cables that are attached to the capstan so that one side pays out cable while the other side pulls in an equal length of cable. With this architecture, three degrees of freedom require a total of six cables extending from the wrist mechanism proximally back along the length of the instrument's main shaft tube to the instrument's proximal mechanical structure. Efficient implementation of a wrist mechanism and proximal mechanical structure can be complicated because the cables must be carefully routed through the tool member, wrist mechanism, and proximal mechanical structure to maintain stability of the wrist throughout the range of motion of the wrist mechanism and to minimize the interactions (or coupling effects) of one rotation axis upon another.

Some known architectures for a manipulator-driven medical device include electrically charged wrist components. In such medical devices, there is a need to protect the tissue being treated from contacting the electrically charged wrist components while maintaining flexibility of the wrist. In such devices, it can be challenging preventing electrically conductive fluid, such as body fluid, from pooling and capacitively coupling with applied energy. In addition, some known medical devices may include a shaft constructed with electrically conductive material such as stainless steel to reduce cost and increase strength. For example, in some known medical devices, the shaft bears a high axial load within the system and presents challenges in providing the strength needed to bear the load while simultaneously meeting the needed size limitations for the shaft and maintaining cost effectiveness. In such devices, however, the need to eliminate electrically conductive fluid contact with the shaft is increased to prevent unintended tissue damage from electrical charge.

In addition, in some minimally invasive surgical instruments having an electrically conductive shaft, electrically conductive liquid (e.g., body fluid, surgical irrigation water) can be drawn via capillary action (e.g., wicking), from outside the instrument's distal end via the tension element (e.g., cable) to inside the instrument shaft. Similarly stated, in some known instruments, a fluid leak path can exist along the instrument cables. In some situations, insufflation pressure can cause an ingress of fluid into the instrument shaft of a medical device. The electrically conductive fluid within the instrument shaft affects the ability to monitor the shaft for electrical charge levels. Although known instruments include mechanisms for distal end tension element sealing that are somewhat effective, improvement is needed, especially for instruments in which the tension element is formed with synthetic/polymeric cable.

Thus, a need exists for improved endoscopic tools that provide a mechanical structure with fluid and electrical isolation features at or near the distal end of the endoscopic tool to facilitate the use of an electrically conductive shaft, such as a stainless steel shaft, by accommodating inner and outer insulation of the shaft and preventing fluid leakage.

### SUMMARY

This summary introduces certain aspects of the embodiments described herein to provide abasic understanding. This summary is not an extensive overview of the inventive subject matter, and it is not intended to identify key or critical elements or to delineate the scope of the inventive subject matter.

In some embodiments, a medical device includes an instrument shaft, a tension element guide, a tension element, and a semisolid sealing substance. The instrument shaft includes a proximal end portion, a distal end portion, and a channel between the proximal end portion and the distal end portion. The tension element guide is at the distal end of the instrument shaft, and includes a proximal seal, a distal seal, and a middle seal between the proximal seal and the distal seal. A sealing substance volume is defined between the proximal seal and the middle seal and holds the sealing substance. The tension element extends from the channel of the instrument shaft, past the proximal seal, through the sealing substance in the sealing substance volume, past the middle seal, past the distal seal, and beyond the tension element guide.

In some embodiments, the medical device includes a spacer at the distal end portion of the shaft having an inner surface. The tension element guide includes an outer surface and the sealing substance volume is defined between the inner surface of the spacer and the outer surface of the tension element guide. In some embodiments, the spacer and the tension element guide are each electrical insulators.

In some embodiments, the middle seal includes an O-ring that extends around the tension element guide and the tension element passes under the O-ring. In some embodiments, the proximal seal includes an O-ring that extends around the tension element guide and the tension element passes under the O-ring.

In some embodiments, the medical device includes a movable component coupled to the distal end portion of the shaft. The movable component has a defined full range of motion. The tension element is coupled to the movable component and translates a first distance to move the movable component through the full range of motion of the movable component. The distal seal is spaced apart from the middle seal by a second distance larger than the first distance.

In some embodiments, the tension element is a braided polymeric cable that includes a set of individual strands. Within the sealing volume, the sealing substance is between the individual strands of the plurality of individual strands such that a capillary action is prevented between the individual strands of the set of individual strands. In some embodiments, the tension element is a first tension element, and the sealing substance volume is a first sealing substance volume. The medical device includes a second tension element and a second sealing substance volume is defined between the proximal seal and the middle seal. The second sealing substance volume holds a portion of the sealing substance and the second tension element extends from the channel of the instrument shaft, past the proximal seal, through the sealing substance in the second sealing substance volume, past the middle seal, past the distal seal, and distally beyond the tension element guide. In some embodiments, the first sealing substance volume and the second sealing substance volume are interconnected within the tension element guide.

In some embodiments, the sealing substance includes a hydrophobic substance. In some embodiments, the tension element guide is a single monolithic piece. In some embodiments, the distal seal is overmolded to the tension element guide.

In some embodiments, the medical device further includes an electrical conductor seal and an electrical conductor that extends through the electrical conductor seal. In some embodiments, the electrical conductor seal is overmolded to the tension element guide. In some embodiments, the instrument shaft is electrically conductive.

In some embodiments, a medical device includes an instrument shaft, a tension element guide, a tension element and a semisolid sealing substance. The instrument shaft includes a proximal end portion, a distal end portion, and a channel between the proximal end portion and the distal end portion. The tension element guide is at the distal end of the instrument shaft and includes a proximal location, a distal location, and a middle location between the proximal location and the distal location. The tension element includes a set of strands. A sealing substance volume is defined between the proximal location of the tension element guide and the middle location of the tension element guide, and the sealing substance volume holds the sealing substance. The tension element extends distally from the channel of the instrument shaft, past the proximal location of the tension element guide, through the sealing substance in the sealing substance volume, past the middle location of the tension element guide, and beyond the distal location of the tension element guide. The tension element is movable proximally and distally within the sealing substance volume and fluid is prevented from traveling in a proximal direction along the tension element from the location distal of the distal location of the tension element guide to a location proximal of the proximal location of the tension element guide by sealing substance from the sealing substance volume being between individual strands of the tension element.

In some embodiments, the medical device includes a proximal seal at the proximal location of the tension element guide, a middle seal at the middle location of the tension element guide, and a distal seal at the distal location of the tension element guide. The middle seal and the distal seal together prevent the sealing substance from migrating from within the sealing substance volume to a location distal of the distal seal. In some embodiments, the middle seal includes an O-ring that extends around the tension element guide and the tension element passes under the O-ring. In some embodiments, the proximal seal includes an O-ring that extends around the tension element guide and the tension element passes under the O-ring.

In some embodiments, the distal seal includes a tension element opening and the tension element opening includes a first taper portion, a second taper portion, and a throat. The throat defines a diameter of the tension element opening. The tension element has a first diameter when the tension element is in an untensioned state and a second diameter when the tension element is in a tensioned state. The first diameter of the tension element is larger than the diameter of the tension element opening, and the second diameter of the tension element is less than the diameter of the tension element opening.

In some embodiments, the medical device includes a movable component coupled to the distal end portion of the shaft and the movable component has a defined full range of motion. The tension element is coupled to the movable component and translates a first distance to move the movable component through the full range of motion of the movable component. The distal seal is spaced apart from the middle seal by a second distance larger than the first distance.

In some embodiments, the medical device includes a spacer at the distal end portion of the shaft and the spacer includes an inner surface. The tension element guide includes an outer surface and the sealing substance volume is defined between the inner surface of the spacer and the outer surface of the tension element guide. In some embodiments, the spacer and the tension element guide are each electrical insulators.

In some embodiments, the distal seal is overmolded to the tension element guide. In some embodiments, the medical device includes an electrical conductor seal and an electrical conductor that extends through the electrical conductor seal. In some embodiments, the instrument shaft is conductive.

Other medical instruments, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of an optional auxiliary unit of the minimally invasive teleoperated surgery system shown in FIG. 1.

FIG. 3 is a perspective view of a user control console of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5A is a diagrammatic illustration of a portion of a medical device according to an embodiment.

FIG. 5B is an enlarged view of a distal end portion of the encircled portion Z in FIG. 5A showing an interior tension element opening of the tension element guide of the medical device of FIG. 5A.

FIG. 5C is a schematic illustration of a portion of a tension element, shown in a first untensioned configuration.

FIG. 5D is a schematic illustration of the portion of the tension element of FIG. 5C, shown in a second tensioned configuration.

FIG. 6 is a schematic illustration of a distal end portion of a medical device according to an embodiment.

FIG. 7 is a schematic illustration of a distal end portion of a medical device according to an embodiment.

FIG. 8 is a schematic illustration of a distal end portion of a medical device according to an embodiment.

FIG. 9 is a perspective view of a medical device according to an embodiment.

FIG. 10 is a perspective view of a distal end portion of the medical device of FIG. 9 with a distal tip cover and outer cover removed.

FIG. 11 is a partial exploded perspective view of a distal end portion of the medical device of FIG. 9.

FIG. 12A is a partial exploded perspective side view of select components of a distal end portion of the medical device of FIG. 9.

FIG. 12B is a top view of the wrist assembly and portion of an electrical conductor of the medical device of FIG. 9.

FIG. 13 is a perspective view of a spacer and tension element guide of the medical device of FIG. 9, shown with tension elements extending therethrough.

FIGS. 14A and 14B are each a distal perspective view of the tension element guide of the medical device of FIG. 9, shown with tension elements and an electrical conductor extending therethrough (FIG. 14B) and shown without the tension elements and electrical conductor (FIG. 14A).

FIG. 14C is a proximal perspective view of the tension element guide of the medical device of FIG. 9 shown with the tension elements.

FIGS. 15A and 15B are each a distal end view of the tension element guide of FIGS. 14A-C, showing tension elements and an electrical conductor extending therethrough (FIG. 15A), and shown without the tension elements and electrical conductor (FIG. 15B).

FIGS. 16 and 17 are each perspective views of the spacer and tension element guide of the medical device of FIG. 9, with the spacer shown transparent for illustration purposes.

FIGS. 18A and 18B are cross-sectional views of a distal end portion of the medical device of FIG. 9 taken along line 18A-18A (offset from a centerline of the shaft) and 18B-18B (along the center line of the shaft), respectively in FIG. 9.

FIG. 19A is a cross-sectional view of a distal end portion medical device taken at a location corresponding to line 19A-19A in FIG. 18B, showing an electrical conductor seal within the tension element guide.

FIG. 19B is a cross-sectional view of a distal end portion of the medical device taken at a location corresponding to line 19B-19B in FIG. 18B, showing the sealing substance volume within the tension element guide.

### DETAILED DESCRIPTION

The embodiments described herein can advantageously be used in a wide variety of grasping, cutting, and manipulating operations associated with minimally invasive surgery. In some embodiments, an end effector of the medical device can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that rotates with reference to a proximal clevis (one DOF).

The medical devices of the present application enable motion in three degrees of freedom (e.g., about a pitch axis, a yaw axis, and a grip axis) using only four tension elements (e.g., cables), thereby reducing the total number of tension elements required, reducing the space required within the shaft and wrist, reducing overall cost, and enables further miniaturization of the wrist and shaft assemblies to promote MIS procedures.

Medical devices described herein can provide for electrical power to be transferred to a wrist assembly and/or tool coupled to the wrist assembly at a distal end of the medical device, such as, for example, a cauterizing or cutting tool. The medical devices have an architecture that provides for protection of tissue from contact with the electrically charged wrist components at the distal end of the medical device, while maintaining flexibility of the wrist. In some embodiments described herein, a medical has a metallic shaft which provides for strength and reduced costs. Such medical devices have an architecture that reduces (and in some instances eliminates) fluid contact with the metallic shaft. For example, a metallic shaft can become capacitively coupled and charged during the application of energy and must be insulated to avoid unintended tissue damage.

As described herein, in some embodiments, a medical device includes a seal at distal end portion of the instrument that protects against escaping insufflation pressure and minimizes fluid leakage around the tension elements (e.g., drive cables) of the medical device without excessive friction. The seal can also prevent body fluids from reaching the electrically charged wrist components and metallic shaft.

In some embodiments, a medical device includes a tension element guide at a distal end of the instrument shaft. The tension element guide includes a proximal seal, a distal seal, and a middle seal and defines a sealing substance volume between the proximal seal and the middle seal that contains a semisolid sealing substance. The tension elements of the medical instrument extend past the proximal seal, through the sealing substance in the sealing substance volume, past the middle seal, past the distal seal, and beyond the tension element guide. This arrangement can also prevent body fluids from reaching the electrically charged wrist components and metallic shaft.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end (i.e., the end manipulated by the user or coupled to the actuation shaft) would be the proximal end of the medical device.

Further, specific words chosen to describe one or more embodiments and optional elements or features are not intended to limit the invention. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

Aspects of the invention are described primarily in terms of an implementation using a da Vinci® surgical system, commercialized by Intuitive Surgical, Inc. of Sunnyvale, California. Examples of such surgical systems are the da Vinci Xi® surgical system (Model IS4000), da Vinci X® Surgical System (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices that are not mechanically grounded in a world reference frame and relatively larger systems that have additional mechanical support that is grounded in a world reference frame.

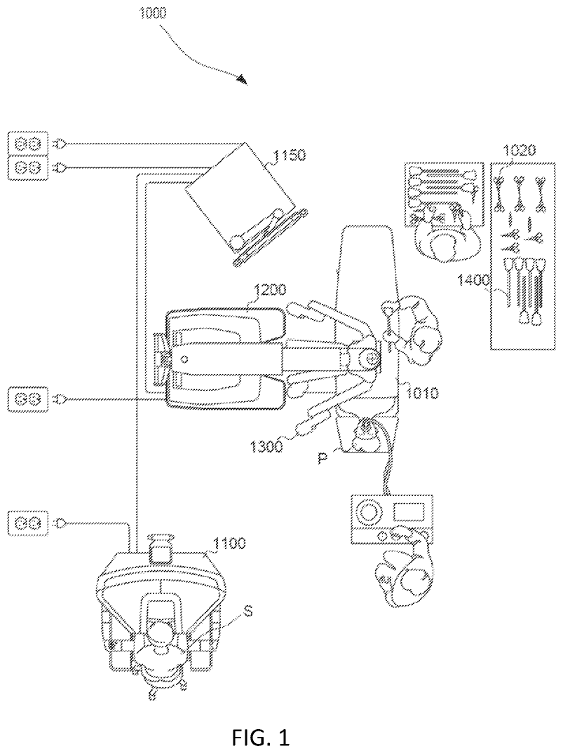

FIG. 1 is a plan view illustration of a teleoperated surgical system 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

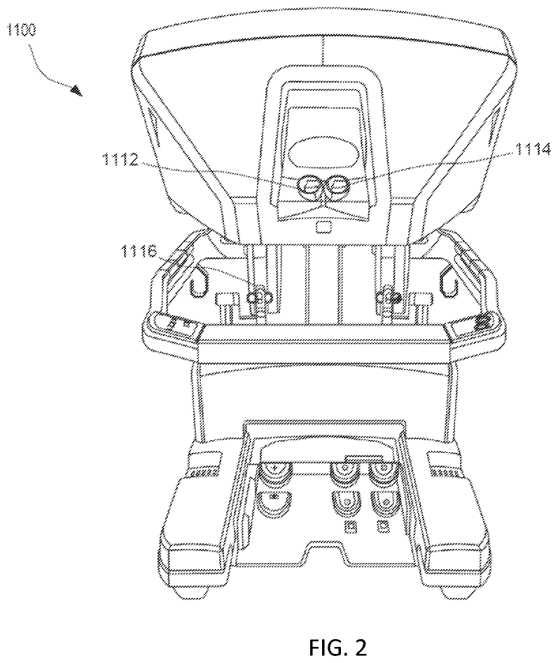

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

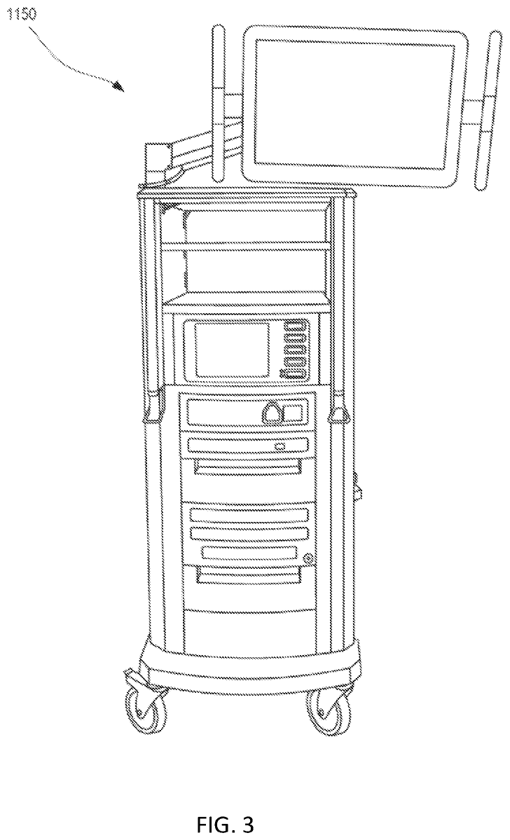

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

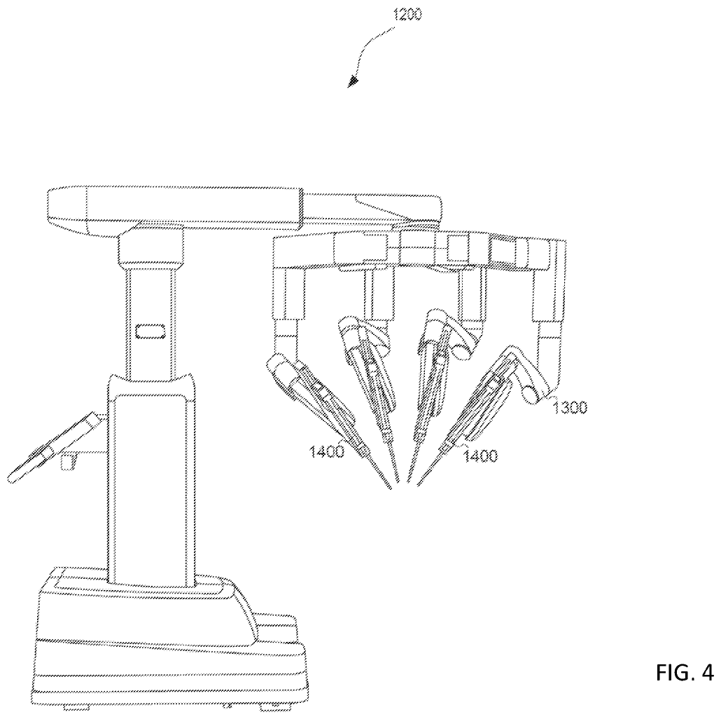

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

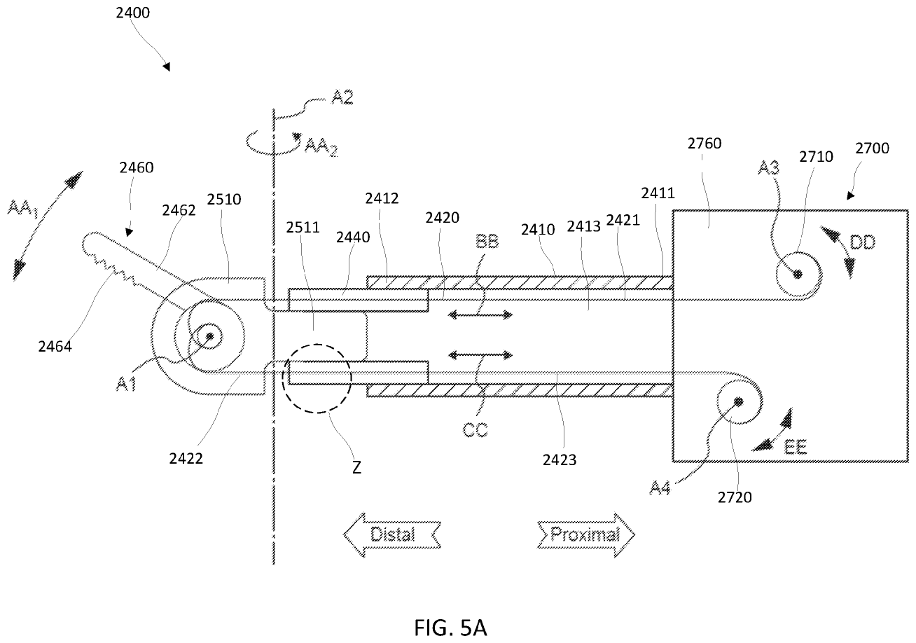

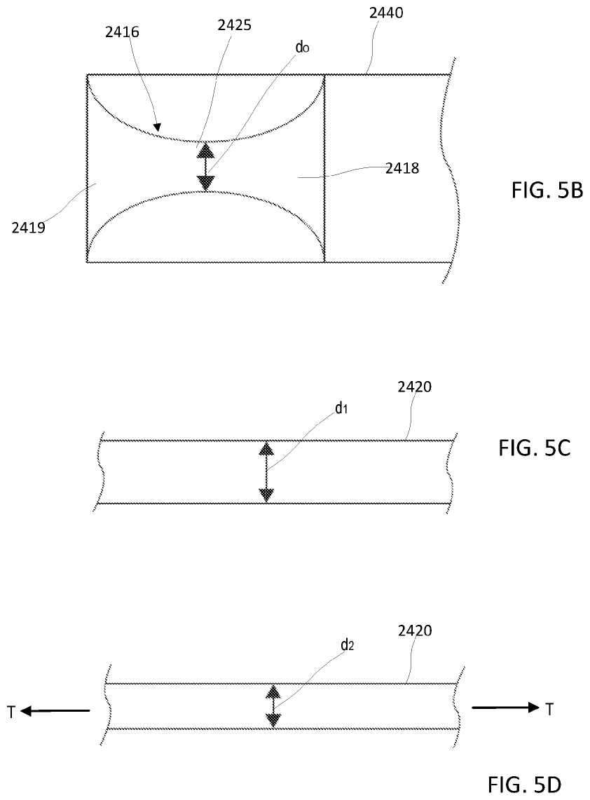

FIGS. 5A-5D are schematic illustrations of a portion of a medical device 2400 according to an embodiment. In some embodiments, the instrument 2400 or any of the components therein are optionally parts of a surgical system that performs surgical procedures, and which can include a manipulator unit, a series of kinematic linkages, a series of cannulas, or the like. The medical device 2400 (and any of the instruments described herein) can be used in any suitable surgical system, such as the MIRS system 1000 shown and described above. The medical device 2400 includes a shaft 2410, a tension element 2420 (e.g., a cable), a tension element guide 2440, a link 2510, an end effector 2460, and a mechanical structure 2700. In many embodiments, mechanical structure 2700 functions to receive one or more motor input forces or torques and mechanically transmit the received forces or torques to move an associated one or more components in the medical device 2400. For example, one or more electric motors in manipulator unit 1200 (described above) provide an input to mechanical structure 2700, which in turn transmits the input to control a medical device 2400 component (e.g., the end effector 2460 or any movable components thereof). In some optional embodiments, mechanical structure 2700 includes one or more motors used to control a medical device 2400 component. In some embodiments, the mechanical structure 2700 (and any of the mechanical structures described herein) can include one or more drive motors to produce the force or torque to move the components of the medical device 2400. In other embodiments, mechanical structure 2700 (and any of the mechanical structures described herein) is devoid of any motors and transmits force or torque from outside the medical device to one or more of the medical device's components. For example, in some embodiments, the mechanical structure 2700 (and any of the mechanical structures described herein) is coupled to a manipulator unit that includes one or more motors that drive an instrument component. The shaft 2410 includes a proximal end portion 2411 and a distal end portion 2412 and defines a channel 2413 that extends lengthwise through the shaft between the proximal and distal end portions.

The tension element guide 2440 is positioned at least partially within the channel 2413 of the shaft 2410 at the distal end portion 2412 of the shaft 2410. The tension element guide 2440 can be electrically insulative and can be formed with an insulative material, such as, for example, a polyphthalamide PPA GF material, or other suitable material. The tension element guide 2440 can be coupled (either directly or indirectly) to an inner surface of the shaft 2410 by a friction fit, adhesive or other suitable coupling methods. The tension element guide 2440 includes one or more tension element openings 2416 (see FIG. 5B) through which the tension element 2420 can pass. More specifically, the tension element 2420 includes a first end, a second end, a first proximal portion 2421, a second proximal portion 2423 and a distal portion 2422. The first end and the second end are each coupled to the mechanical structure 2700, the first proximal portion 2421 and the second proximal portion 2423 extend through the channel 2413 of the shaft 2410, and the distal portion 2422 extends through the tension element opening 2416 and is coupled to the end effector 2460 as described in more detail below.

In some embodiments, the tension element guide 2440 includes one or more seal portions (not shown in FIG. 5A) to limit liquid leakage into the shaft 2410, and also limit leakage of insufflation gas out of the interior body cavity of the patient being treated via the medical device 2400. For example, in some embodiments, a seal portion can provide a seal between an inner surface of the shaft 2410 and an outer perimeter of the tension element guide 2440, and/or a seal between an outer surface of a proximal coupling protrusion 2511 of the link 2510 and an inner surface of the tension element guide 2440.

In some embodiments, the tension element openings 2416 are shaped and sized to also limit liquids and/or insufflation gas from leaking through the openings 2416 while also allowing the tension element 2420 to move within the openings 2416. More specifically, as shown in FIG. 5B, the tension element openings 2416 have an hourglass shape that has a first tapered portion 2418 at a first end of the openings 2416, a second tapered portion 2419 at a second end, and a throat portion 2425 having a smaller diameter dₒ than a diameter of the first tapered portion 2418 and the second tapered portion 2419. The hourglass shape of the tension element openings 2416 and the diameter dₒ provide for a tight fit to the tension element 2420 when the tension element is under tension to prevent liquids and/or gas leakage through the openings 2416. For example, the tension element 2420 can be formed with a polymeric material such that the tension element 2420 can deform under tension (as shown by the arrows T in FIG. 5D). As shown in FIG. 5C, the tension element 2420 has a first nominal or untensioned state that has a diameter d₁ and a second, tensioned state with a diameter d₂, as shown in FIG. 5D. The ratio between the diameter d₁ and the diameter d₂ can be, for example, 1.2:1 and 1.6:1. In some embodiments, the diameter of the tension element 2420 can be reduced between 0-30% of its first nominal diameter. The diameter d₁ is larger than the diameter dₒ of the tension element openings 2416, and the diameter d₂ of the tension element 2420 is smaller than the diameter dₒ. Thus, the tension element 2420 can be passed through the tension element openings 2416 when in the tensioned state and when the tension element 2420 is untensioned, the tension element 2420 will return to its untensioned, nominal diameter d₁ and form a tight fit to the throat portion 2425 of the tension element openings 2416. The tension element openings 2416 are configured to protect against escaping insufflation pressure and minimize fluid leakage around the tension elements 2420 of the medical device without excessive friction.

In some embodiments, a distal end portion of the tension element 2420 and/or a portion of the tension element guide 2440 that defines the tension element openings 2416 can include an oil coating. In some embodiments, a distal end portion of the tension element 2420 and/or a portion of the tension element guide 2440 (or any seals therein) that defines the tension element openings 2416 can include a hydrophobic material.

In some embodiments, the medical device 2400 can include a spacer (not shown in FIG. 5A), such as spacer 5900 and 6900, and an outer cover (not shown in FIG. 5A), such as outer cover 6910 described herein. The spacer can be positioned within the passageway 2413 of the shaft 2410 and extend distally of the shaft 2410, and the tension element guide 2440 can be positioned within an interior passageway of the spacer. The tension element guide 2440 can function in the same manner as described above and provide a first sealing portion to the inner surface of the spacer and a second sealing portion via the tension element openings 2416. The outer cover can surround the exterior of the shaft 2410 and extend distally of the distal end of the shaft 2410. In this manner, the tension element guide 2440, the spacer and outer cover can isolate the conductive shaft 2410 from electrical energy. For example, the shaft 2410 can become capacitively coupled and charged during the application of energy, and the spacer, outer cover and, the tension element guide 2440 (or any seals therein) can isolate the shaft 2410 to prevent fluid contact with the shaft 2410 and to avoid unintended damage to tissue during treatment using the medical device 2400.

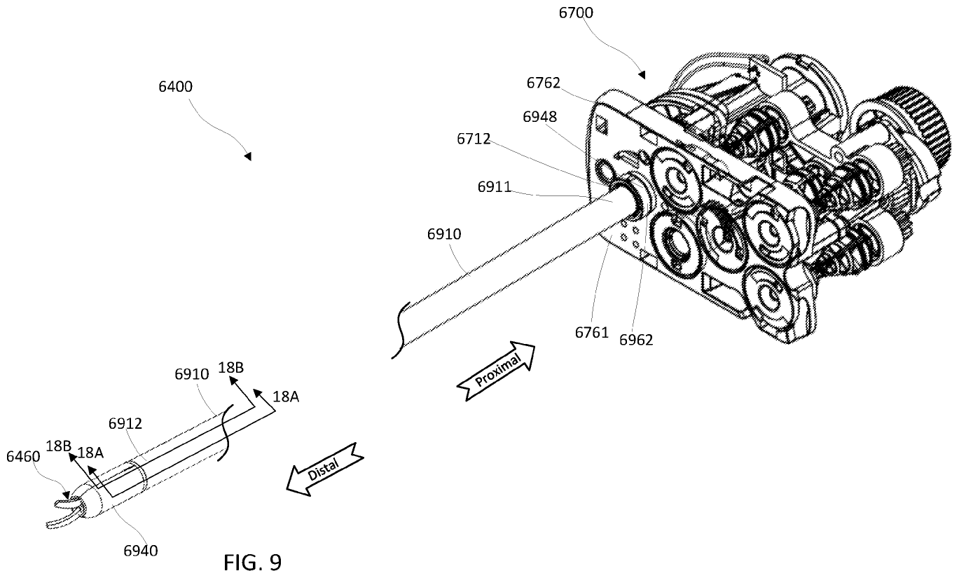

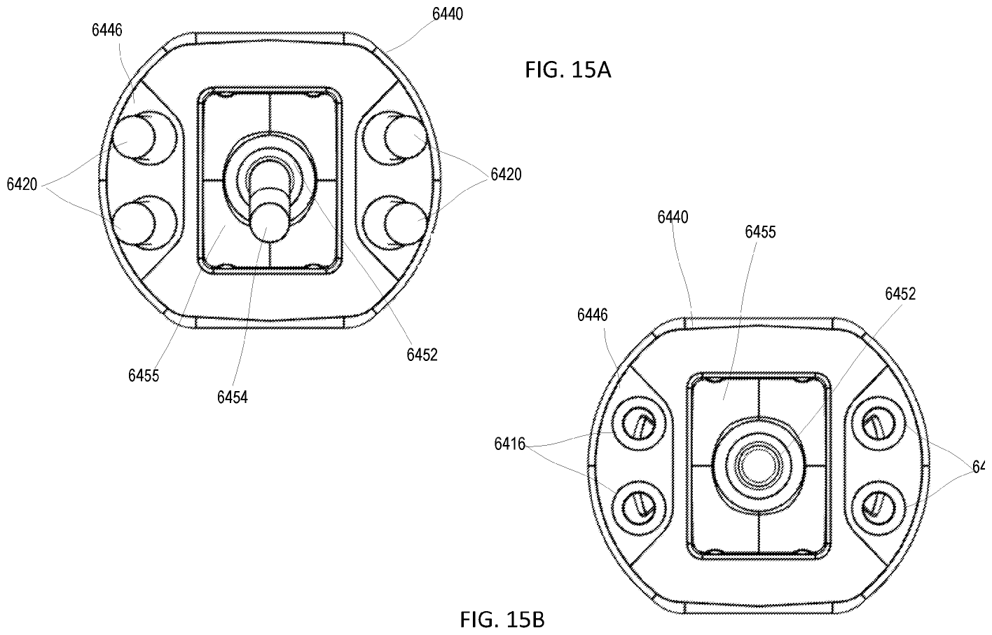

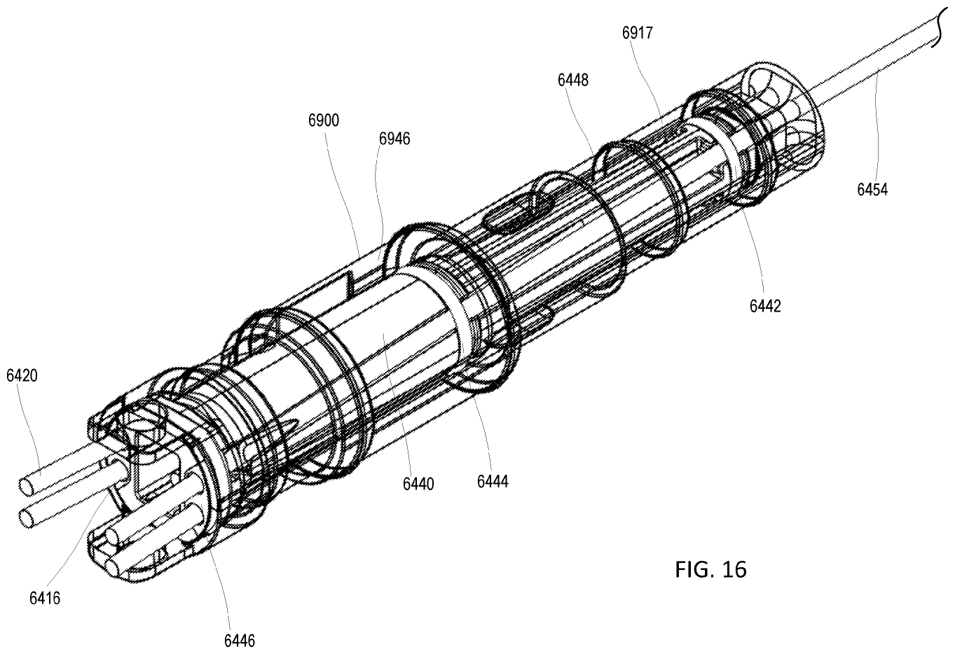

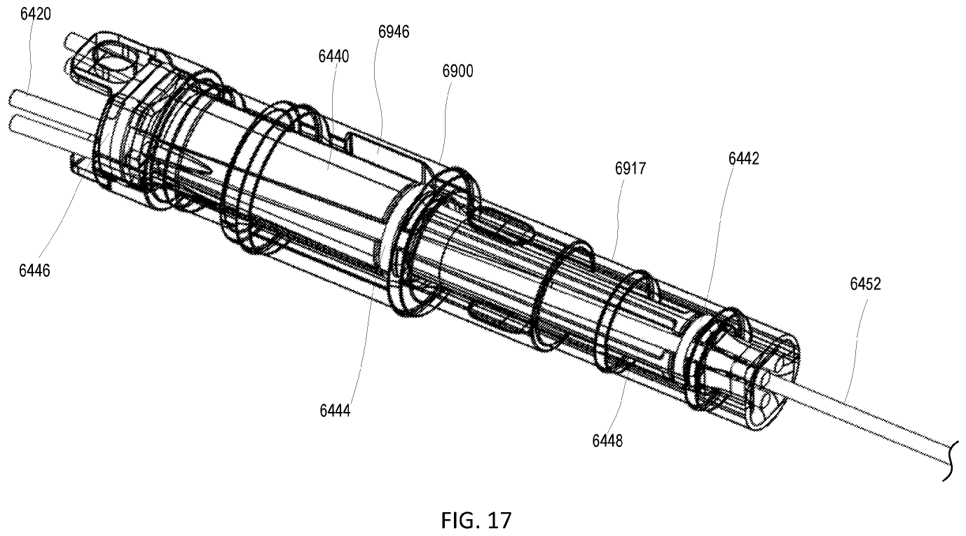

The end effector 2460 is rotatably coupled to the distal end portion 2412 of the shaft 2410 and includes at least one tool member 2462 (that functions as a movable component). The medical device 2400 is configured such that movement of the first proximal portion 2421 and the second proximal portion 2423 of the tension element 2420 produces movement of the tool member 2462 about a first axis of rotation A1 (which functions as the yaw axis; the term yaw is arbitrary), in a direction of arrows AA₁. In some embodiments, the medical device 2400 can include a wrist assembly including one or more links 2510 that couples the end effector 2460 to the distal end portion 2412 of the shaft 2410. In such an embodiment, movement of the first proximal portion 2421 and the second proximal portion 2423 of the tension element 2420 can also produce movement of the wrist assembly about a second axis of rotation (which functions as the pitch axis; the term pitch is arbitrary) or both movement of the wrist assembly and the end effector 2460. An embodiment with a wrist assembly is described herein with reference to FIGS. 9-19B.

The tool member 2462 includes a contact portion 2464, configured to engage or manipulate a target tissue during a surgical procedure. For example, in some embodiments, the contact portion 2464 can include an engagement surface that functions as a gripper, cutter, tissue manipulator, or the like. In other embodiments, the contact portion 2464 can be an energized portion of the tool member that is used for cauterization or electrosurgical procedures. The end effector 2460 is operatively coupled to the mechanical structure 2700 such that the tool member 2462 rotates relative to shaft 2410 about the first axis of rotation A1 in the direction of the arrow AA₁. In this manner, the contact portion 2464 of the tool member 2462 can be actuated to engage or manipulate a target tissue during a surgical procedure. The tool member 2462 (or any of the tool members or movable components described herein) can be any suitable medical tool member. Moreover, although only one tool member 2462 is shown, in other embodiments, the medical device 2400 can include two or more moving tool members that cooperatively perform gripping or shearing functions.

The mechanical structure 2700 includes a chassis 2760, a first capstan 2710, and a second capstan 2720. The chassis 2760 provides the structural support for mounting or supporting and aligning the components of the mechanical structure 2700. For example, openings, protrusions, mounting brackets and the like can be defined in or on chassis 2760. In some embodiments, the chassis 2760 can include multiple portions, such as an upper chassis and a lower chassis. In some embodiments, a housing can optionally enclose at least a portion of the chassis 2760. The first capstan 2710 is mounted to the mechanical structure 2700 (e.g., within the housing 2760) via a first capstan support member (not shown). For example, the first capstan support member can be a mount, shaft, or any other suitable support structure to secure the first capstan 2710 to the mechanical structure 2700.

The second capstan 2720 is mounted to the mechanical structure 2700 (e.g., within the housing 2760) via a second capstan support member (not shown). For example, the second capstan support member can be a mount, shaft, or any other suitable support structure to secure the second capstan 2720 to the mechanical structure 2700. The first capstan 2710 can be operable to be rotated about an axis A3 in a direction DD, as shown in FIG. 5A. The second capstan 2720 can be operable to be rotated about an axis A4 parallel to the axis A3.

The tension element 2420 is routed from the mechanical structure 2700 to the end effector 2460 and then back to mechanical structure 2700, and the first end of the tension element 2420 is coupled to the first capstan 2710 and the second end of the tension element 2420 is coupled to the second capstan 2720 of the mechanical structure 2700. The tension element 2420 extends from the first capstan 2710 along the shaft 2410, and the distal portion 2422 of the tension element 2420 passes through the tension element opening 2416 of the tension element guide 2440 (or any seals therein) and is coupled to the end effector 2460. Although the tension element 2420 is shown extending within an interior passageway 2413 of the shaft 2410 in FIG. 5A, in other embodiments, the tension element 2420 can be routed exterior to the shaft 2410. The tension element 2420 extends from the end effector 2460 along or within the passageway 2413 of the shaft 2410 and the second end of the tension element 2410 is coupled to the second capstan 2720 of the mechanical structure 2700. In other words, the two ends of a single tension element (e.g., 2420) are coupled to and actuated by two separate capstans of the mechanical structure 2700.

More specifically, the two ends of the tension element 2420 that are associated with opposing directions of a single degree of freedom are connected to two independent drive capstans 2710 and 2720. This arrangement, which is generally referred to as an antagonist drive system, allows for independent control of the movement of (e.g., pulling in or paying out) each of the ends of the tension element 2420. The mechanical structure 2700 produces movement of the tension element 2420, which operates to produce the desired articulation movements (pitch, yaw, or grip) at the end effector 2460. Accordingly, as described herein, the mechanical structure 2700 includes components and controls to move a first portion of the tension element 2420 via the first capstan 2710 in a first direction (e.g., a proximal direction) and to move a second portion of the tension element 2420 via the second capstan 2720 in a second opposite direction (e.g., a distal direction). The mechanical structure 2700 can also move both the first portion of the tension element 2420 and the second portion of the tension element 2420 in the same direction. In this manner, the mechanical structure 2700 can maintain the desired tension within the tension elements to produce the desired movements at the end effector 2460.

In other embodiments, however, any of the medical devices described herein can have the two ends of the tension element wrapped about a single capstan. This alternative arrangement, which is generally referred to as a self-antagonist drive system, operates the two ends of the tension element using a single drive motor.

In addition, in some alternative embodiments, the tension element 2420 includes two tension element segments, with each tension element segment having a distal end portion that is coupled to the end effector 2460 and a proximal end portion wrapped about a capstan-either separate capstans as in the antagonist drive arrangement or a single common capstan in the self-antagonist drive arrangement. Descriptions herein referring to the use of a single tension element 2420 incorporate the similar use of two separate tension element segments.

With the tension element 2420 coupled to the mechanical structure 2700 and to the end effector 2460, rotational movement produced by the first capstan 2710 causes the first proximal portion 2421 of the tension element 2420 to move in a direction BB (e.g., proximally or distally depending on the direction of rotation), as shown in FIG. 5A. Similarly, rotational movement produced by the second capstan 2720 causes the second proximal portion 2423 of the tension element 2420 to move in the direction CC (e.g., proximally or distally depending on the direction of rotation), as shown in FIG. 5A. Said another way, the first capstan 2710 can be operable to produce rotational movement about the axis A3, and the second capstan 2720 can similarly be operable to produce rotational movement about an axis A4 parallel to the axis A3. Thus, the first capstan 2710 can rotate in the direction of arrows DD and the second capstan 2720 can rotate in the direction of arrows EE in FIG. 5A. For example, when the first capstan 2710 rotates about the axis A3 in a first direction (clockwise or counter-clockwise), the second capstan 2720 rotates about the axis A4 in either the same or the opposite direction (clockwise or counter-clockwise). Thus, as one of the capstans 2710, 2720 pays out the tension element 2420, the other of the capstans 2710, 2720 pays in the tension element 2420. Depending on how the tension elements are routed to the various capstans, it doesn't matter what direction each of the individual capstans rotates as long as the desired individual tension element pay-in or pay-out is performed to perform the desired end effector motion—grip, yaw, or pitch—either alone or in combination.

With each of the ends of the tension element 2420 coupled to a separate capstan, the movement of a first portion of the tension element 2420 can be directly controlled by one capstan (e.g., first capstan 2710) and movement of a second portion of the tension element 2420 can be directly controlled by the other capstan (e.g., second capstan 2720). Thus, the control of motion of the end effector 2460 in one direction is controlled by one capstan, and the control of motion of the end effector 2460 in the other direction is controlled by the other capstan. In this antagonist system, however, when the first capstan 2710 is controlling motion (i.e., applying tension to pull in the first proximal portion 2421 of the tension element 2420), the second proximal portion 2423 of the tension element is also under tension applied by the second capstan 2720. Maintaining tension applied by the non-driving capstan (i.e., the second capstan 2720) allows the non-driving capstan to immediately function as the driving capstan with no hysteresis in end effector control. The differing levels of tension applied by each capstan can also lead to improved control of the overall movement of the tension element. Thus, better control of the overall movement of the end effector 2460 can be achieved. For example, accurate rotation in yaw around axis A1 can be controlled. The first capstan 2710 can be actuated to produce a rotational movement about the axis A3 in the direction of the arrow DD such that the first proximal portion 2421 of the tension element is moved in a first direction along arrows BB. Simultaneously, the second capstan 2720 can be actuated to produce rotational movement about an axis parallel to the axis A3 in an opposite direction as the first capstan 2710 such that the second proximal end portion 2723 of the tension element 2420 is moved in an opposite direction as the first proximal portion 2423 along arrows CC. Thus, the opposite movement of the first proximal portion 2421 and the second proximal portion 2423 causes the end effector 2460 to rotate (via the tension element 2420 connection to the end effector 2460) about the rotational axis A1 (e.g., yaw movement).

In a similar way, accurate rotation in pitch around a second axis A2 (e.g., pitch; orthogonal to the yaw axis A1 described above) can be controlled. As described above, the first capstan 2710 can be actuated to produce a rotational movement about the axis A3 in the direction of the arrow DD, while simultaneously the second capstan 2720 can be actuated to produce rotational movement about the axis A4 parallel to the axis A3 in the direction of the arrow EE such that the first proximal portion 2421 of the tension element 2420 and the second proximal portion 2423 of the tension element 2420 are moved together in the same direction (along arrows BB and CC, respectively). The movement of the first proximal portion 2421 and the second proximal portion 2423 in the same direction causes the end effector 2460 (or a wrist mechanism) to rotate (via the tension element 2420 connection to the end effector 2460) about a second rotation axis A2 in the direction of arrow AA₂ (e.g., pitch movement). Persons of skill in the art will understand that this action controls rotation around the second axis A2 in a first direction, and a similar action by an additional tension element (or segments)(not shown) controls rotation around the second axis A2 in a second direction opposite the first direction. Thus, an antagonistic control relationship between the portions of the tension element 2420 acting together and the additional tension element is used to accurately control end effector rotation in pitch. Alternatively, a resiliency such as a spring may be used to act against portions of the tension element 2420 to urge rotation around the second axis A2 in a direction opposite to the direction urged by portions of tension element 2420. Thus, the combination of the first capstan 2710, the second capstan 2720, and the single tension element 2420 are operable to control the end effector 2460 of medical device 2400 in at least 2 DOFs (e.g., pitch and yaw).

The tension element 2420, and any of the tension elements described herein can have any suitable structure and can be made from any suitable materials. In some embodiments, a tension element 2420 can be a cable that is constructed from multiple woven or braided filaments, wires, or strands. In some embodiments, a tension element 2420 can be a band or other flexible structure that is constructed from one or more laminates or layers. In some embodiments, a tension element 2420 can include a rod or other rigid structure. In some embodiments, the tension element 2420 can be a polymeric braided construction. For example, in some embodiments, any of the tension elements described herein can be formed from a polymer, such as an ultra-high molecular weight polyethylene (UHMWPE) fiber. In some embodiments, any of the tension elements described herein can be constructed from a single monofilament. In other embodiments, any of the tension elements described herein can be constructed from multiple cofilament strands, laid or woven (or both), or thermally fused, or otherwise combined to form the tension element. In some embodiments, the tension element 2420 or any of the tension elements described herein can include an optional outer sheath, coating, or other surface treatment to increase the frictional characteristics of the tension element.

In some embodiments, the tension element 2420 and any of the tension elements described herein can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the tension element 2420 to the capstan 2710 and end effector 2460. Such ability can also improve slip characteristics (e.g., help prevent the tension element from slipping) during operation of the medical device. Such materials may or may not need a coating or other surface treatment to increase the frictional characteristic.

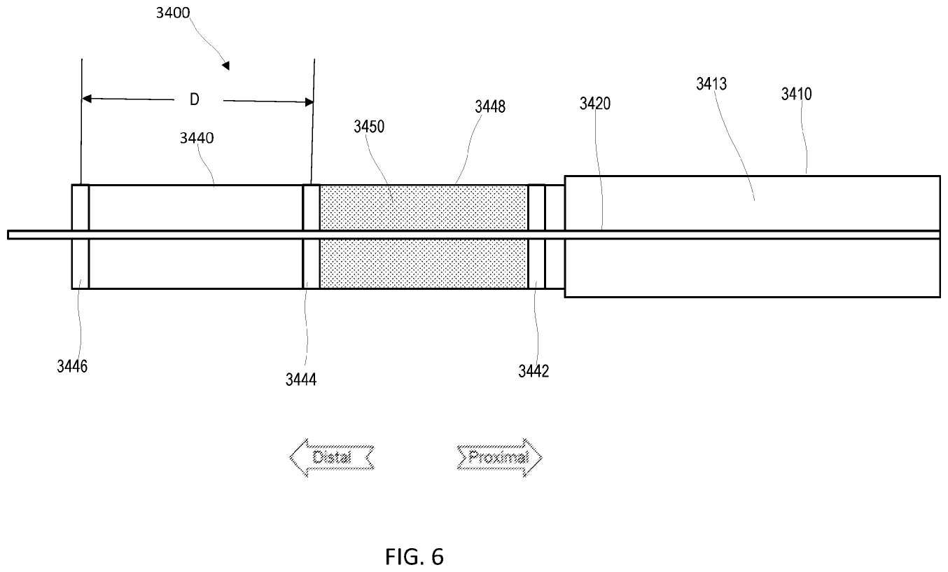

FIG. 6 is a schematic illustration of a distal end portion of a medical device 3400 according to an embodiment. In some embodiments, the medical device 3400 or any of the components therein are optionally parts of a surgical system that performs surgical procedures, and which can include a manipulator unit, a series of kinematic linkages, a series of cannulas, or the like. The medical device 3400 (and any of the instruments described herein) can be used in any suitable surgical system, such as the MIRS system 1000 shown and described above. The medical device 3400 includes a shaft 3410, a tension element guide 3440, and a tension element 3420. Although not shown, the medical device 3400 can also include other components of a medical device as a described herein, such as for example, a proximal mechanical structure, an end effector, and a spacer (described below, for example, with respect to the medical device 6400). The shaft 3410 includes a shaft channel 3413 within which the tension element guide 3440 can be at least partially positioned. The shaft 3410 can be formed, for example, with an electrically conductive material such as stainless steel.

The tension element 3420 can be coupled between a movable component (not shown in FIG. 6) (e.g., wrist assembly, end effector, or tool member) and a proximal mechanical structure (not shown) of the medical device 3400 as described above for medical device 2400, and function in the same manner. The tension element 3420, and any of the tension elements described herein can be made from any suitable materials. In some embodiments, the tension element 3420 can be a polymeric braided construction. For example, in some embodiments, any of the tension elements described herein can be formed from a polymer, such as an ultra-high molecular weight polyethylene (UHMWPE) fiber. In some embodiments, any of the tension elements described herein can be constructed from a single monofilament. In other embodiments, any of the tension elements described herein can be constructed from multiple cofilament strands, laid or woven (or both), or thermally fused, or otherwise combined to form the tension element. In some embodiments, the tension element 3420 or any of the tension elements described herein can include an optional outer sheath, coating, or other surface treatment to increase the frictional characteristics of the tension element.

In some embodiments, the tension element 3420 and any of the tension elements described herein can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the tension element 3420 to a capstan of the mechanical structure (e.g., 2710 of the proximal mechanical structure 2700 described above) and end effector such as end effector 2460. Such ability can also improve slip characteristics (e.g., help prevent the tension element from slipping) during operation of the medical device. Such materials may or may not need a coating or other surface treatment to increase the frictional characteristic.

The tension element guide 3440 is coupled to a distal end portion of the shaft 3410 and includes a proximal seal 3442, a middle seal 3444, and a distal seal 3446. In some embodiments, the proximal seal 3442 and the middle seal 3444 are each an O-ring positioned on an outer surface of the tension element guide 3440. In some embodiments, the O-rings can be formed with a silicone material. The tension element guide 3440 also defines a sealing substance volume 3448 between the proximal seal 3442 and the middle seal 3424. For example, the sealing substance volume 3448 is defined by an outer surface of the tension element guide 3440 between the proximal seal 3442 and the middle seal 3444. The sealing substance volume 3448 can be, for example, one or more grooves within the tension element guide 3440, a recessed portion defined by the tension element guide 3440, a scalloped surface of the tension element guide 3440, or any other suitable volume. A sealing substance 3450 is within the sealing substance volume 3448. The sealing substance 3450 provides a seal to prevent fluid (e.g., gas and/or liquid) from traveling from a location distal of the middle seal 3444 to a location proximal of the proximal seal 3442. For example, during use of the medical device 3400, electrically conductive liquid (e.g., body fluid, surgical irrigation water) is susceptible to being drawn via capillary action (e.g., wicking) from outside the distal end of the medical device 3400 via the tension element 3420 to inside the shaft 3410. In some situations, fluid can enter the medical device 3400 via insufflation pressure. The sealing substance prevents such capillary action and prevents fluids from reaching the electrically conductive shaft 3410 (e.g., formed with a material such as stainless steel) and causing unintended damage to tissue.

The sealing substance 3450 can be, for example, a human biocompatible semisolid sealing substance such as, grease, lubricant, gel, etc. In some embodiments, the sealing substance 3450 is NuSil™ MED-422 Silicone Fluid. In some embodiments, the sealing substance 3450 is a hydrophobic substance.

The tension element 3420 extends distally from the shaft channel 3413 of the shaft 3410, past the proximal seal 3442, through the sealing substance 3450 in the sealing substance volume 3448, past the middle seal 3444, past the distal seal 3446, and distally beyond the tension element guide 3440. In some embodiments, the tension element 3420 passes under the proximal seal 3442 and the middle seal 3444. Said another way, in some embodiments, the tension element can be between a surface of the tension element guide 3440 and each of the proximal seal 3442 and the middle seal 3444. In other embodiments, however, the tension element 3420 extend through one or both of the proximal seal 3442 or the middle seal 3444. In some embodiments, the tension element 3420 is a braided polymeric cable as described above that includes multiple individual strands. With the tension element 3420 passing through the sealing substance 3450, the sealing substance 3450 becomes deposited between the individual strands of the tension element 3450 such that a capillary action (e.g., wicking) is prevented between the individual strands of the tension element 3420. This prevents fluids from traveling in a proximal direction along the tension element 3420 from a location distal of the tension element guide 3440 to a location proximal of the proximal seal 3442.

As described herein, the medical device 3400 can include a movable component (not shown in FIG. 6) (e.g., a wrist assembly, a tool member, etc. as described above for medical device 2400) coupled to a distal end portion of the tension element guide 3440. The movable component can have a defined full range of motion as described above for medical device 2400. The tension element 3420 is coupled to the movable component and translates a preset distance within the shaft 3410 and the sealing substance volume 3448 (e.g., proximally and distally) to move the movable component through the full range of motion of the movable component. Similarly stated, the medical device is configured such that the tension element 3420 has a travel distance that corresponds to movement of the movable member through its full range of motion. The distal seal 3446 is spaced apart from the middle seal 3444 by a distance D that is larger than the preset distance that the tension element 3420 travels. The spacing between the distal seal 3446 and the middle seal 3444 prevents sealing substance 3450 deposited on the tension element 3420 from being transported distally beyond the distal seal 3446.

In some embodiments, the distal seal 3446 is overmolded to the tension element guide 3440. In some embodiments, the distal seal 3446 can be formed with, for example, Santoprene™ thermoplastic vulcanizates (TPVs), a thermoplastic polyurethane (TPU) material, or other suitable material. The distal seal 3446 includes one or more tension element openings (not shown), one of which the tension element 3420 passes through. The tension element openings can be shaped and sized to also limit fluids (e.g., liquids and/or insufflation gas) from leaking through the openings while also allowing the tension element 3420 to move within the openings. For example, the tension element openings can be shaped and sized as described and shown for tension element openings 2416.

In some embodiments, the tension element guide 3440 can be electrically insulative and can be formed with an insulative material, such as, for example, a polyphthalamide PPA GF material, or other suitable material. The tension element guide 3440 can be a single monolithically constructed component or can include multiple components coupled together. In some embodiments, the tension element guide 3440 includes a first sealing substance volume (e.g., 3448) and a second sealing substance volume (not shown) and the sealing substance 3450 is within both the first and second sealing substance volumes. In some embodiments, the first and second sealing substance volumes are interconnected. In other words, the sealing substance 3450 can move between the first and second sealing substance volumes. In some embodiments, the first and second sealing substance volumes are not interconnected (i.e., the first and second sealing substance volumes are closed from each other). In such embodiments, the medical device 3400 can include a second tension element (not shown) that extends from the channel 3413 of the shaft 3410, past the proximal seal 3442, through the sealing substance 3450 in the second sealing substance volume, past the middle seal 3444, past the distal seal 3446, and distally beyond the tension element guide 3440.

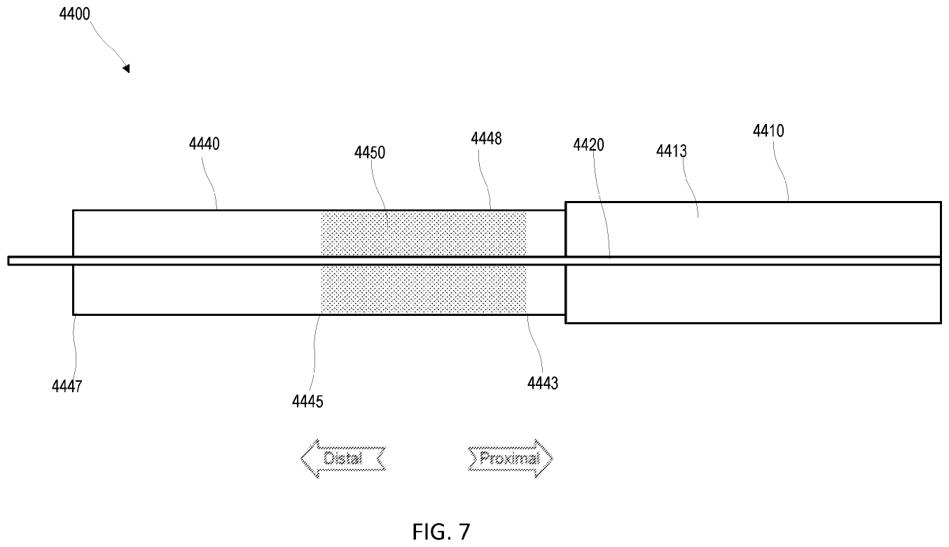

FIG. 7 is a schematic illustration of a distal end portion of a medical device 4400 according to an embodiment. In some embodiments, the medical device 4400 or any of the components therein are optionally parts of a surgical system that performs surgical procedures, and which can include a manipulator unit, a series of kinematic linkages, a series of cannulas, or the like. The medical device 4400 (and any of the instruments described herein) can be used in any suitable surgical system, such as the MIRS system 1000 shown and described above. The medical device 4400 includes a shaft 4410, a tension element guide 4440, and a tension element 4420. Although not shown, the medical device 4400 can also include other components of a medical device as a described herein, such as for example, a proximal mechanical structure, an end effector, a spacer, as described herein (for example, with respect to the medical device 6400). The shaft 4410 includes a shaft channel 4413 within which the tension element guide 4440 can be at least partially positioned. The shaft 4410 can be formed, for example, with an electrically conductive material such as stainless steel.

The tension element 4420 can be coupled between a movable component (e.g., a wrist assembly, end effector, or tool member) (not shown in FIG. 7) and a proximal mechanical structure (not shown in FIG. 7) of the medical device 4400 as described above for medical device 2400, and function in the same manner. The tension element 4420, and any of the tension elements described herein can be made from any suitable materials. In some embodiments, the tension element 4420 can be a polymeric braided construction. For example, in some embodiments, any of the tension elements described herein can be formed from a polymer, such as an ultra-high molecular weight polyethylene (UHMWPE) fiber. In other embodiments, any of the tension elements described herein can be constructed from multiple cofilament strands, laid or woven (or both), or thermally fused, or otherwise combined to form the tension element. In some embodiments, the tension element 4420 or any of the tension elements described herein can include an optional outer sheath, coating, or other surface treatment to increase the frictional characteristics of the tension element.

In some embodiments, the tension element 4420 and any of the tension elements described herein can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the tension element 4420 to a capstan of the mechanical structure (e.g., 2710 of the proximal mechanical structure 2700 described above) and end effector (e.g., end effector 2460 described above). Such ability can also improve slip characteristics (e.g., help prevent the tension element from slipping) during operation of the medical device. Such materials may or may not need a coating or other surface treatment to increase the frictional characteristic.

The tension element guide 4440 is coupled to a distal end portion of the shaft 4410 and includes a proximal location 4443, a middle location 4445, and a distal location 4447. The tension element guide 4440 also defines a sealing substance volume 4448 between the proximal location 4443 and the middle location 4445. For example, the sealing substance volume 4448 is defined by an outer surface of the tension element guide 4440 between the proximal location 4443 and the middle location 4445. The sealing substance volume 4448 can be, for example, one or more grooves within the tension element guide 4440, a recessed portion defined by the tension element guide 4440, a scalloped surface of the tension element guide 4440, or any other suitable volume. For example, in some embodiments, the proximal location 4443 and the middle location 4445 include ribs or shoulders (not shown) that define the sealing substance volume 4448. A sealing substance 4450 is within the sealing substance volume 4448. The sealing substance 4450 provides a seal to prevent fluid (e.g., gas and/or liquid) from traveling from a location distal of the middle seal 4445 to a location proximal of the proximal location 4443. For example, during use of the medical device 4400, electrically conductive liquid (e.g., body fluid, surgical irrigation water) is susceptible to being drawn via capillary action (e.g., wicking) from outside the distal end of the medical device 4400 via the tension element 4420 to inside the shaft 4410. In some situations, fluids can enter the medical device 4400 via insufflation pressure. The sealing substance prevents such capillary action and prevents fluids from reaching the electrically conductive shaft 4410 (e.g., formed with a material such as stainless steel) and causing unintended damage to tissue.

The sealing substance 4450 can be, for example, a human biocompatible semi-solid sealing substance such as, grease, lubricant, gel, etc. In some embodiments, the sealing substance 4450 is NuSil™ MED-422 Silicone Fluid. In some embodiments, the sealing substance 4450 is a hydrophobic substance.

The tension element 4420 extends distally from the shaft channel 4413 of the shaft 4410, past the proximal location 4443, through the sealing substance 4450 in the sealing substance volume 4448, past the middle location 4445, past the distal location 4447 of the tension element guide 4440, and distally beyond the tension element guide 4440. The tension element 4420 is a braided polymeric cable as described herein that includes multiple individual strands. With the tension element 4420 passing through the sealing substance 4450, the sealing substance 4450 becomes deposited between the individual strands of the tension element 4450 such that a capillary action (e.g., wicking) is prevented between the individual strands of the tension element 4420. This prevents fluids from traveling in a proximal direction along the tension element 4420 from a location distal of the tension element guide 4440 to a location proximal of the proximal location 4443. Thus, this arrangement limits the likelihood of having such fluid contact the shaft 4410.

As described herein, the medical device 4400 can include a movable component (not shown in FIG. 7) (e.g., a wrist assembly, a tool member, etc. as described above for medical device 2400) coupled to a distal end portion of the tension element guide 4440. The movable component can have a defined full range of motion as described above for medical device 2400. The tension element 4420 is coupled to the movable component and translates a preset distance within the shaft 4410 and the sealing substance volume 4448 (e.g., proximally and distally) to move the movable component through the full range of motion of the movable component.

In some embodiments, the tension element guide 4440 can include any or all of a proximal seal (not shown in FIG. 7) at the proximal location 4443, a middle seal (not shown in FIG. 7) at the middle location 4445, and a distal seal (not shown in FIG. 7) at the distal location 4447. In such an embodiment, the distal seal can be spaced apart from the middle seal by a distance larger than a preset distance that the tension element 4420 travels within the tension element guide 4440 such that the sealing substance 4450 deposited on the tension element 4420 is not transported distally beyond the distal seal, as described above for medical device 3400.

In some embodiments, the distal seal is overmolded to the tension element guide 4440. In some embodiments, the distal seal can be formed with, for example, Santoprene™ thermoplastic vulcanizates (TPVs), a thermoplastic polyurethane (TPU) material, or other suitable material. The distal seal can include one or more tension element openings (not shown), one of which the tension element 4420 passes through. The tension element openings can be shaped and sized to also limit fluids (e.g., liquids and/or insufflation gas) from leaking through the openings while also allowing the tension element 4420 to move within the openings. For example, the tension element openings can be shaped and sized as described and shown for tension element openings 2416.

In some embodiments, the tension element guide 4440 can be electrically insulative and can be formed with an insulative material, such as, for example, a polyphthalamide PPA GF material, or other suitable material. The tension element guide 4440 can be a single monolithically constructed component or can include multiple components coupled together. In some embodiments, the tension element guide 4440 includes a first sealing substance volume (e.g., 4448) and a second sealing substance volume (not shown) and the sealing substance 4450 is within both the first and second sealing substance volumes. In some embodiments, the first and second sealing substance volumes are interconnected. In other words, the sealing substance 4450 can move between the first and second sealing substance volumes. In some embodiments, the first and second sealing substance volumes are not interconnected (i.e., the first and second sealing substance volumes are closed from each other). In such embodiments, the medical device 4400 can include a second tension element (not shown) that extends from the channel 4413 of the shaft 4410, past the proximal location 4443, through the sealing substance 4450 in the second sealing substance volume, past the middle location 4445, past the distal location 4447, and distally beyond the distal end of the tension element guide 4440.

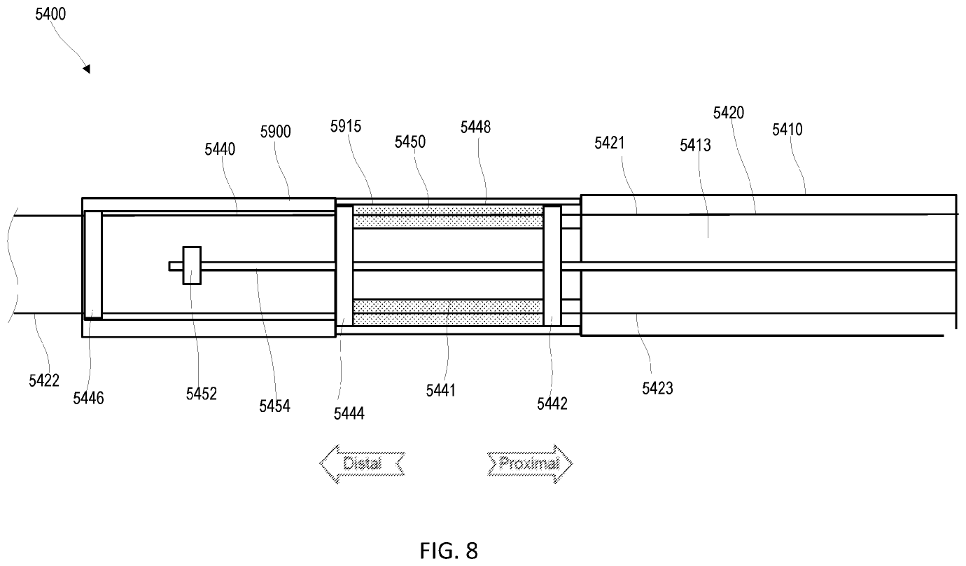

FIG. 8 is a schematic illustration of a distal end portion of a medical device 5400 according to an embodiment. In some embodiments, the medical device 5400 or any of the components therein are optionally parts of a surgical system that performs surgical procedures, and which can include a manipulator unit, a series of kinematic linkages, a series of cannulas, or the like. The medical device 5400 (and any of the instruments described herein) can be used in any suitable surgical system, such as the MIRS system 1000 shown and described above. The medical device 5400 includes a shaft 5410, a tension element guide 5440, and a tension element 5420, a spacer 5900, and an electrical conductor 5454. Although not shown, the medical device 5400 can also include other components of a medical device as a described herein, such as for example, a proximal mechanical structure, and an end effector. The shaft 5410 includes a shaft channel 5413 within which the tension element guide 5440 can be at least partially positioned. The shaft 5410 can be formed, for example, with an electrically conductive material such as stainless steel.

The tension element 5420 can be coupled between a movable component (e.g., wrist assembly, end effector, or tool member) (not shown) and a proximal mechanical structure (not shown) of the medical device 5400 as described above for medical device 2400, and function in the same manner. The tension element 5420 includes a first end, a second end, a first proximal portion 5421, a second proximal portion 5423 and a distal portion 5422. The first end and the second end are each coupled to the proximal mechanical structure (not shown) of the medical device 5400, the first proximal portion 5421 and the second proximal portion 5423 extend through the channel 5413 of the shaft 5410, and the distal portion 5422 extends through the tension element guide 5440 is coupled to the movable component (not shown) of the medical device 5400.

The tension element 5420, and any of the tension elements described herein can be made from any suitable materials. In some embodiments, the tension element 5420 can be a polymeric braided construction. For example, in some embodiments, any of the tension elements described herein can be formed from a polymer, such as an ultra-high molecular weight polyethylene (UHMWPE) fiber. In some embodiments, any of the tension elements described herein can be constructed from a single monofilament. In other embodiments, any of the tension elements described herein can be constructed from multiple cofilament strands, laid or woven (or both), or thermally fused, or otherwise combined to form the tension element. In some embodiments, the tension element 5420 or any of the tension elements described herein can include an optional outer sheath, coating, or other surface treatment to increase the frictional characteristics of the tension element.

In some embodiments, the tension element 5420 and any of the tension elements described herein can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the tension element 5420 to a capstan of the mechanical structure (e.g., 2710 of the proximal mechanical structure 2700 described above) and end effector (e.g., end effector 2460 described above). Such ability can also improve slip characteristics (e.g., help prevent the tension element from slipping) during operation of the medical device. Such materials may or may not need a coating or other surface treatment to increase the frictional characteristic.

The tension element guide 5440 is coupled to a distal end portion of the shaft 5410 and includes a proximal seal 5442, a middle seal 5444, and a distal seal 5446. In some embodiments, either or both of the proximal seal 5442 or the middle seal 5444 are each an O-ring positioned on an outer surface 5441 of the tension element guide 5440. In some embodiments, the O-rings can be formed with a silicone material. The tension element guide 5440 also defines a sealing substance volume 5448 between the proximal seal 5442 and the middle seal 5444. For example, the sealing substance volume 5448 is defined by an outer surface 5441 of the tension element guide 5440 and an inner surface 5915 of the spacer 5900, between the proximal seal 5442 and the middle seal 5444. The sealing substance volume 5448 can be, for example, one or more grooves within the tension element guide 5440 or the spacer 5900, a recessed portion defined by the tension element guide 5440 or the spacer 5900, a scalloped surface of the tension element guide 5440 or the spacer 5900, or any other suitable volume. For example, in some embodiments, either or both of the tension element guide 5440 or the spacer 5900 include ribs or shoulders (not shown) that define the sealing substance volume 5448. A sealing substance 5450 is within the sealing substance volume 5448. The sealing substance 5450 provides a seal to prevent fluid (e.g., gas and/or liquid) from traveling from a location distal of the middle seal 5444 to a location proximal of the proximal seal 5442. For example, during use of the medical device 5400, electrically conductive liquid (e.g., body fluid, surgical irrigation water) is susceptible to being drawn via capillary action (e.g., wicking) from outside the distal end of the medical device 5400 via the tension element 5420 to inside the shaft 5410. In some situations, fluids can enter the medical device 5400 via insufflation pressure. The sealing substance prevents such capillary action and prevents fluids from reaching the electrically conductive shaft 5410 (e.g., formed with a material such as stainless steel) and causing unintended damage to tissue.

The sealing substance 5450 can be, for example, a human biocompatible semi-solid sealing substance such as, grease, lubricant, gel, etc. In some embodiments, the sealing substance 5450 is NuSil™ MED-422 Silicone Fluid. In some embodiments, the sealing substance 5450 is a hydrophobic substance.

The tension element 5420 extends distally from the shaft channel 5413 of the shaft 5410, past the proximal seal 5442, through the sealing substance 5450 in the sealing substance volume 5448, past the middle seal 5444, past the distal seal 5446, and distally beyond the tension element guide 5440. In some embodiments, the tension element 5420 passes under the proximal seal 5442 and the middle seal 5444. Said another way, in some embodiments, the tension element 5420 can be between a surface of the tension element guide 5440 and each of the proximal seal 5442 and the middle seal 5444. In other embodiments, however, the tension element 5420 extends through one or both of the proximal seal 5442 or the middle seal 5444. In some embodiments, the tension element 5420 is a braided polymeric cable as described above that includes multiple individual strands. With the tension element 5420 passing through the sealing substance 5450, the sealing substance 5450 becomes deposited between the individual strands of the tension element 5450 such that a capillary action (e.g., wicking) is prevented between the individual strands of the tension element 5420. This prevents fluids from traveling in a proximal direction along the tension element 5420 from a location distal of the tension element guide 5440 to a location proximal of the proximal seal 5442.

As described herein, the medical device 5400 can include a movable component (not shown in FIG. 6) (e.g., a wrist assembly, a tool member, etc. as described above for medical device 2400) coupled to a distal end portion of the tension element guide 4440. The movable component can have a defined full range of motion as described above for medical device 2400. The tension element 5420 is coupled to the movable component and translates a preset distance within the shaft 5410 and the sealing substance volume 5448 (e.g., proximally and distally) to move the movable component through the full range of motion of the movable component. The distal seal 5446 is spaced apart from the middle seal 5444 by a distance larger than the preset distance that the tension element 5420 travels. The spacing between the distal seal 5446 and the middle seal 5444 prevents sealing substance 5450 deposited on the tension element 5420 from being transported distally beyond the distal seal 5446.

In some embodiments, the distal seal 5446 is overmolded to the tension element guide 5440. In some embodiments, the distal seal 5446 can be formed with, for example, Santoprene™ thermoplastic vulcanizates (TPVs), a thermoplastic polyurethane (TPU) material, or other suitable material. The distal seal 5446 includes one or more tension element openings (not shown), through which the tension element 5420 passes through. The tension element openings can be shaped and sized to also limit fluids (e.g., liquids and/or insufflation gas) from leaking through the openings while also allowing the tension element 5420 to move within the openings. For example, the tension element openings can be shaped and sized as described and shown for tension element openings 2416.

The tension element guide 5440 also includes an electrical conductor seal 5452 within an interior of the tension element guide 5440. In some embodiments, the electrical conductor seal 5452 is between the middle seal 5444 and the distal seal 5446. In some embodiments, the electrical conductor seal 5452 is formed with, for example, Santoprene™ thermoplastic vulcanizates (TPVs), a thermoplastic polyurethane (TPU) material, or other suitable material. In some embodiments, the electrical conductor seal 5452 is overmolded to (or within) the tension element guide 5440. The electrical conductor 5454 extends through the shaft 5410, past the proximal seal 5442, past the middle seal 5444, and through the electrical conductor seal 5452. The electrical conductor seal 5452 prevent fluids from traveling from a location distal of the electrical conductor seal 5452 to a location proximal of the electrical conductor seal 5452.

The spacer 5900 surrounds the tension element guide 5440 and can be electrically insulative and formed with an insulative material, such as, for example, a polyphthalamide PPA GF material, or other suitable material. The spacer 5900 extends within the channel 5413 of the shaft 541- and extends distally of a distal end of the shaft 5410. As described above, the inner surface 5915 of the spacer 5900 and the outer surface 5441 of the tension element guide 5440 collectively define the sealing substance volume 5448.

In some embodiments, the tension element guide 5440 can be electrically insulative and can be formed with an insulative material, such as, for example, a polyphthalamide PPA GF material, or other suitable material. The tension element guide 5440 can be a single monolithically constructed component or can include multiple components coupled together. In some embodiments, the tension element guide 5440 includes a first sealing substance volume (e.g., 5448) and a second sealing substance volume (not shown) and the sealing substance 5450 is within both the first and second sealing substance volumes. In some embodiments, the first and second sealing substance volumes are interconnected. In other words, the sealing substance 5450 can move between the first and second sealing substance volumes. In some embodiments, the first and second sealing substance volumes are not interconnected (i.e., the first and second sealing substance volumes are closed from each other). In such embodiments, the medical device 5400 can include a second tension element (not shown) that extends from the channel 5413 of the shaft 5410, past the proximal seal 5442, through the sealing substance 5450 in the second sealing substance volume 5448, past the middle seal 5444, past the distal seal 5446, and distally beyond the tension element guide 5440.

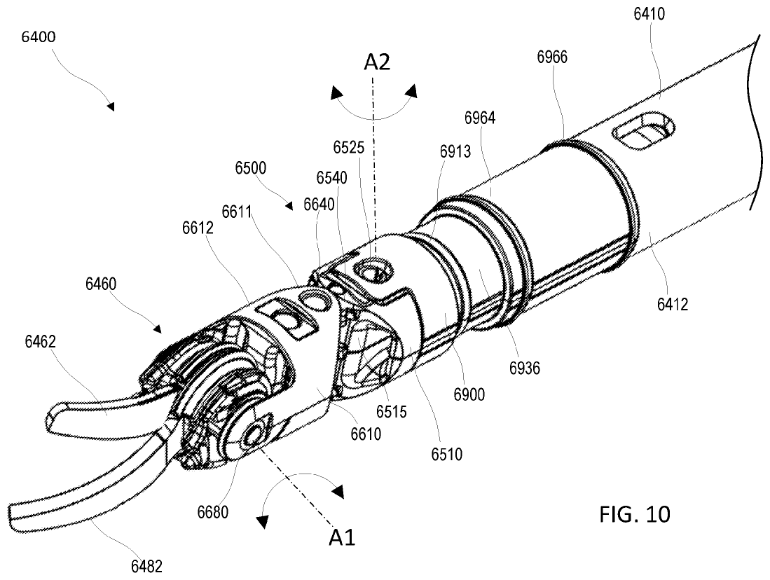

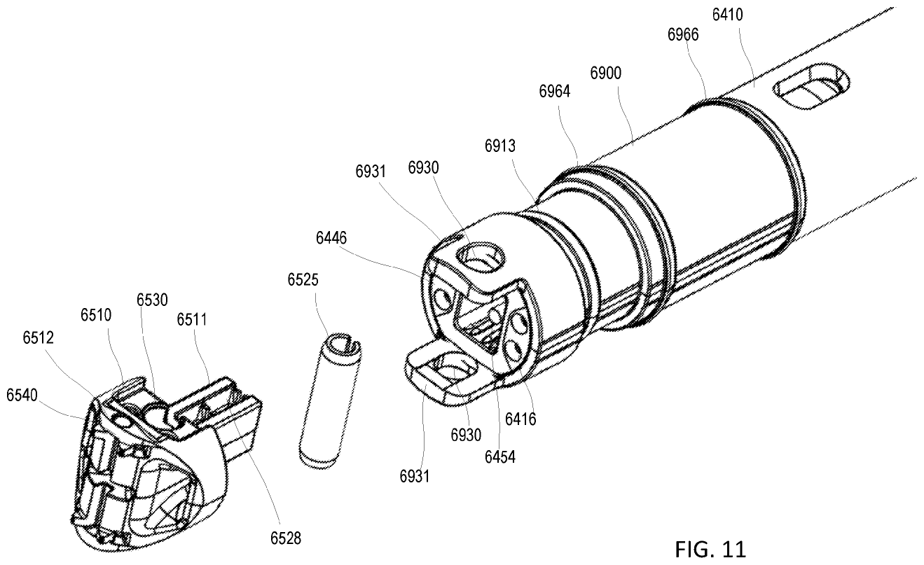

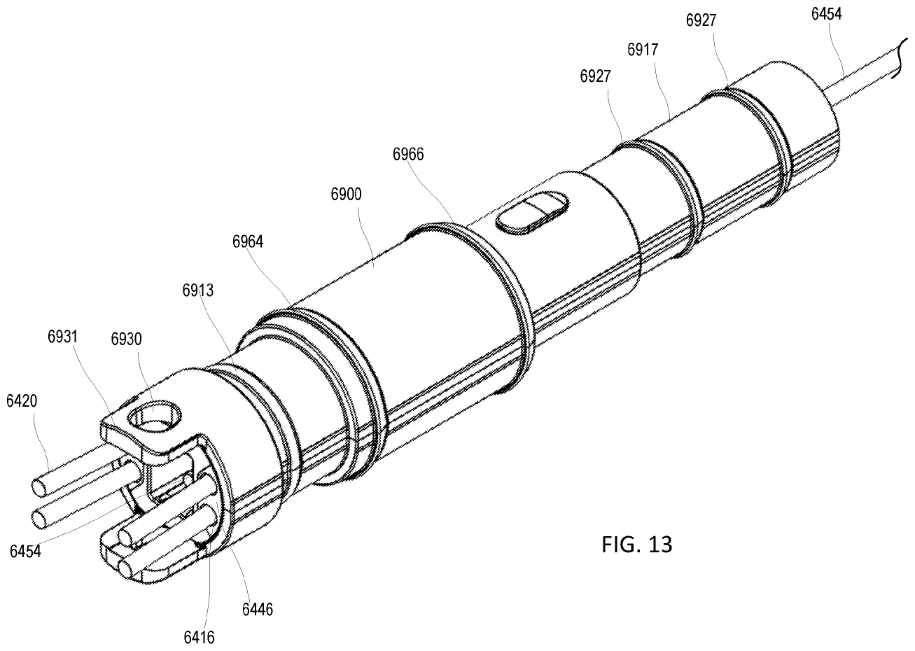

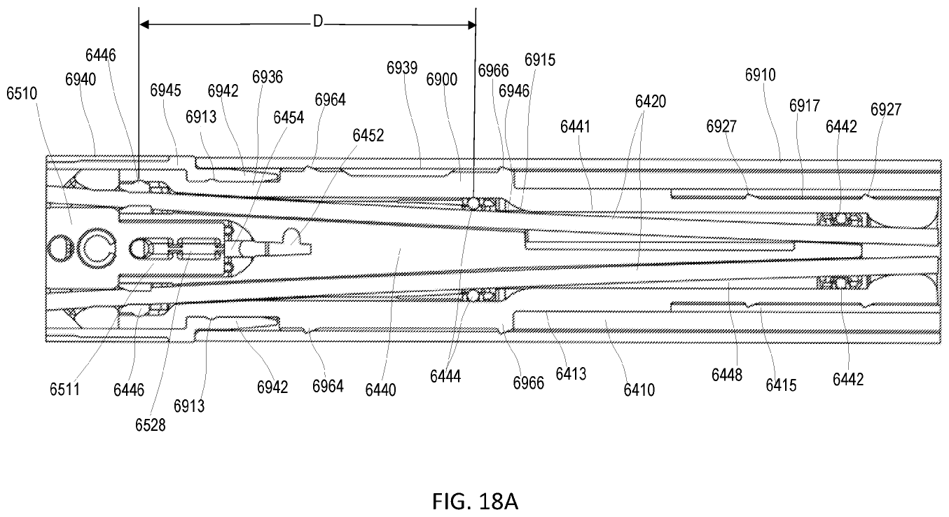

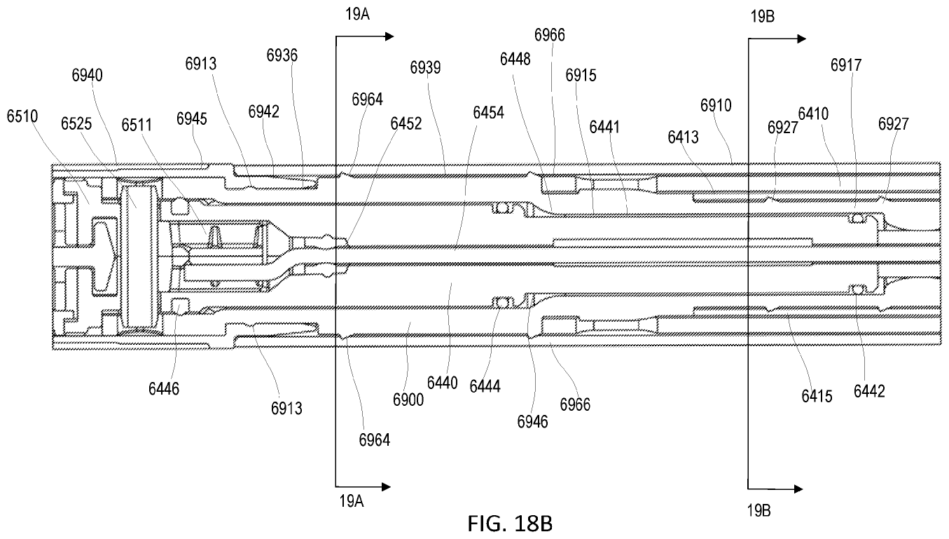

FIGS. 9-19B are various views of an instrument 6400, according to an embodiment. In some embodiments, the instrument 6400 or any of the components therein are optionally parts of a surgical system that performs surgical procedures, and which can include a manipulator unit, a series of kinematic linkages, a series of cannulas, or the like. The instrument 6400 (and any of the instruments described herein) can be used in any suitable surgical system, such as the MIRS system 1000 shown and described above. The instrument 6400 includes a proximal mechanical structure 6700, a shaft 6410 (see, e.g., FIGS. 10 and 11), an inner insulative cover 6415 (see, e.g., FIGS. 18A and 18B), an outer insulative cover 6910 (see e.g., FIG. 9), a distal wrist assembly 6500 (see FIG. 10), a distal end effector 6460 (see FIG. 10), a distal tip cover 6940, an inner tip cover 6945 (see, e.g., FIGS. 18A and 18B-), a tension element guide 6440, and a spacer 6900. The instrument 6400 also includes one or more tension elements 6420 (see FIGS. 13, 14B, 14C, 15A, 16-18B) that couple the proximal mechanical structure 6700 to the distal wrist assembly 6500 and end effector 6460. The instrument 6400 is configured such that movement of one or more of the tension elements 6420 produces rotation of the end effector 6460 about a first axis of rotation A1 (see FIG. 10, which functions as a yaw axis, the term yaw is arbitrary), rotation of the wrist assembly 6500 about a second axis of rotation A2 (see FIG. 10, which functions as a pitch axis), a cutting rotation of the tool members of the end effector 6460 about the first axis of rotation A1, or any combination of these movements. Changing the pitch or yaw of the instrument 6400 can be performed by manipulating the tension elements in a similar manner as that described above for the medical device 2400.

The shaft 6410 can be any suitable elongated shaft that is coupled to the wrist assembly 6500 and to the mechanical structure 6700 (either directly or via intermediate structure, such as via the spacer 6900 or tension element guide 6440, as described herein). Specifically, the shaft 6410 includes a proximal end (not shown) that is coupled to the mechanical structure 6700, and a distal end 6412 (see FIGS. 10, 11, 18A and 18B) that is coupled to the wrist assembly 6500 (e.g., a proximal link of the wrist assembly 6500) via the spacer 6900 and tension element guide 6440, as described herein. The inner insulative cover 6415 (see FIGS. 18A and 18B) is disposed within a passageway 6413 (see FIGS. 18A and 18B) of the shaft 6410. The shaft 6410 and the inner insulative cover 6415 collectively define a passageway, through which the tension elements and other components (e.g., charged electrical wires, ground wires, or the like) can be routed from the mechanical structure 6700 to the wrist assembly 6500. The shaft 6410 can be formed, at least in part with, for example, an electrically conductive material such as stainless steel. The inner insulative cover 6415 can be formed with a thermoplastic polyester elastomer, for example, Hytrel® material, or one or more other insulative materials.

The outer cover 6910 can also be formed with a thermoplastic polyester elastomer, for example, Hytrel® material, or one or more other insulative materials. The outer cover 6910 surrounds an outer surface of a portion of the spacer 6900 and an outer surface of at least a portion of the shaft 6410.

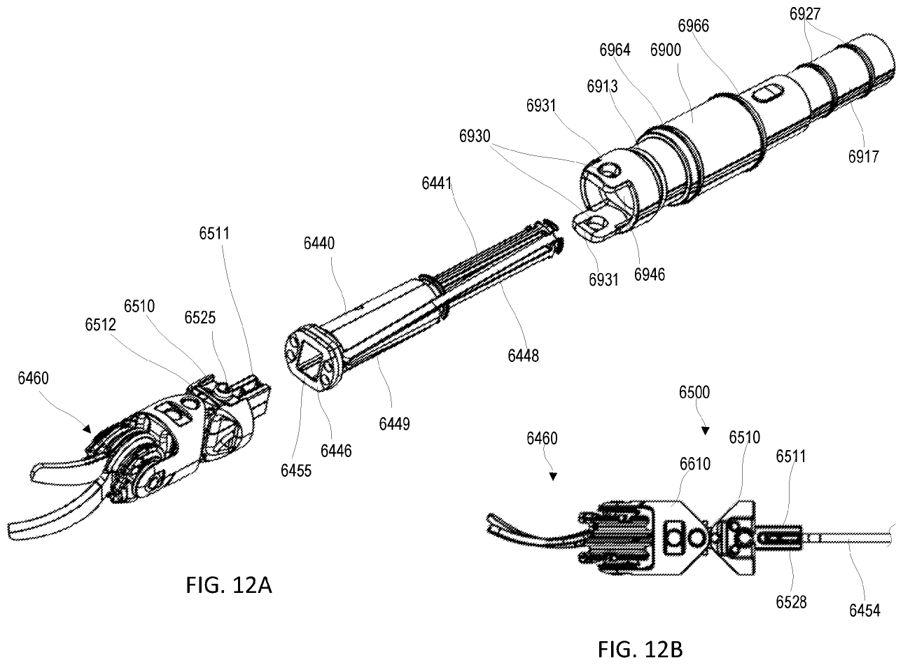

Referring to FIG. 10, the wrist assembly 6500 (also referred to as a joint assembly) includes a first link 6510, a second link 6610 and a third link 6515. The first link 6510 has a proximal portion 6511 (see, FIGS. 11 and 12) and a distal end portion 6512. The proximal portion 6511 includes a coupling protrusion that is coupled to the tension element guide 6440, as described in more detail below. The proximal portion 6511 can be coupled to the tension element guide 6440 via any suitable mechanism. For example, in some embodiments, the proximal portion 6511 can be matingly disposed within an interior region 6455 of the tension element guide 6440 (e.g., via an interference fit). In some embodiments, the proximal portion 6511 can include one or more protrusions, recesses, openings, or connectors that couple the proximal portion 6511 to the tension element guide 6440. The proximal portion 6511 of the first link 6510 is coupled to the spacer 6900 with a spring pin 6525 that is received through openings 6930 defined in flanges 6931 of the spacer 6900 and opening 6530 of the proximal portion 6511 (see, e.g., FIGS. 11 and 12). In some embodiments, the proximal portion 6511 can be welded, glued, or fused to the tension element guide 6440 and/or spacer 6900. At least some portions of the wrist assembly 6500 are formed with a metallic material and are used in the delivery of electrical energy to tool members 6462 and 6482 of the end effector 6460. For example, the first link 6510 and the second link 6610 can be formed with a metallic material. The coupling protrusion of the proximal portion 6511 of the link 6510 includes an interior region 6528 that can receive an electrical conductor 6454 to electrically couple the electrical conductor 6454 to the proximal link 6511, as shown in FIGS. 12B and 18A.

The distal end portion 6512 of the wrist assembly 6500 includes a joint portion 6540 that is rotatably coupled to a mating joint portion 6640 of the second link 6610 as described in more detail below. The second link 6610 has a proximal portion 6611 and a distal end portion 6612. The proximal portion 6611 includes a joint portion 6640 that is rotatably coupled to the joint portion 6540 of the first link 6510 to form the wrist assembly 6500 having the second axis of rotation A2 about which the second link 6610 rotates relative to the first link as shown in FIG. 10. The wrist assembly 6500 can include any suitable coupling mechanisms. In this embodiment, the first link 6510 is coupled to the third link 6515 via a pinned joint and the second link 6610 is coupled to the third link 6515 via a pinned joint. In this manner, the third link 6515 maintains the coupling between the first link 6510 and the second link 6610 during rotation of the second link 6610 relative to the first link 6510.

As shown in FIG. 10, the end effector 6460 is coupled to the second link 6610. More specifically, the distal end portion 6612 of the second link 6610 includes a connector 6680 that is coupled to the end effector 6460 such that the end effector 6460 (e.g., tool members 6462 and 6482 of the end effector) rotates relative to the wrist assembly 6500 about the first axis of rotation A1 (see, e.g., FIG. 10). The second axis of rotation A2 is non-parallel to the first axis of rotation A1. The first axis A1 also functions as a cutting axis as tool members rotate in opposition to each other as described in more detail below. Thus, the medical device 6400 provides at least three degrees of freedom (i.e., yaw motion about the first axis of rotation A1, pitch rotation about the second axis of rotation A2, and a cutting motion about the first axis of rotation A1). In alternative embodiments, depending on the type of end effector, rather than a cutting motion, the end effector can provide motion for other actions, such as, for example, grasping, cauterizing, etc. about the first axis of rotation A1. The connector 6680 can be any suitable connector to rotatably couple the end effector 6460 to the wrist assembly 6500. For example, in some embodiments, the first link 6510 can include a clevis and a pin, such as the pinned joints shown and described in U.S. Pat. No. 9,204,923 (filed Jul. 16, 2008), entitled “Medical Device Electronically Energized Using Drive Cables,” which is incorporated herein by reference in its entirety.

As shown in FIGS. 9 and 10, the end effector 6460 includes the first tool member 6462 and the second tool member 6482. The first tool member 6462 and the second tool member 6482 each include a contact portion (not identified) configured to engage or manipulate a target tissue during a surgical procedure. In this embodiment, the contact portion of the first tool member 6462 and the second tool member 6482 include an engagement surface that functions as a cutter (e.g., a cutting blade). The cutting blades can be any suitable blades, such as those shown and described in copending U.S. Provisional Patent Application Ser. No. 63/234,662, entitled “Surgical Instrument Shears,” the disclosure of which is incorporated herein by reference in its entirety. The first tool member 6462 and the second tool member 6482 can also be energized to use for cauterization or electrosurgical procedures. The end effector 6460 can be operatively coupled to the mechanical structure 6700 such that the tool members 6462 and 6482 rotate about the first axis of rotation A1. In this manner, the contact portions of the tool member 6462 and the contact portion of the tool member 6482 can be actuated to engage or manipulate a target tissue during a surgical procedure.

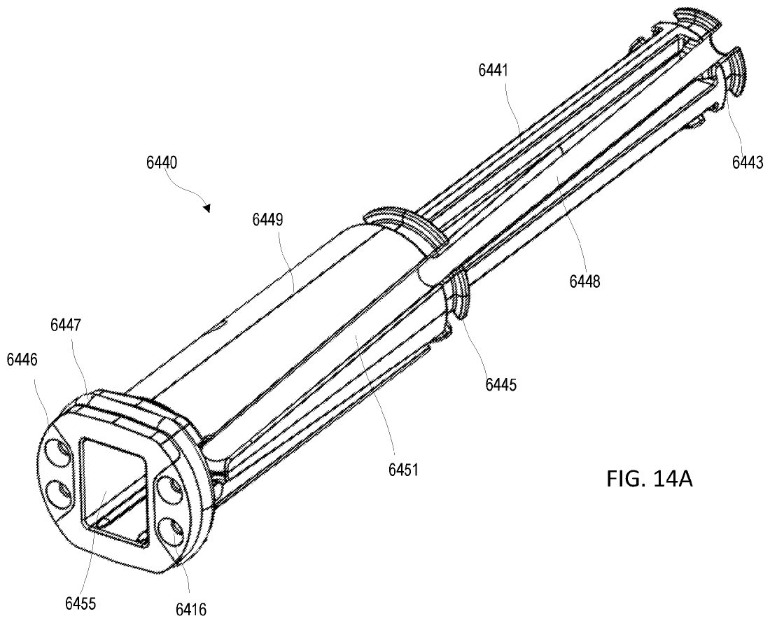

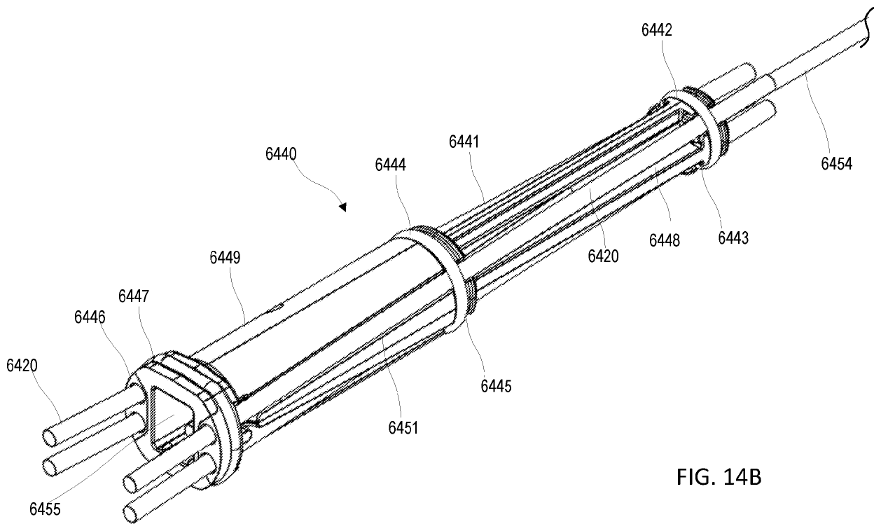

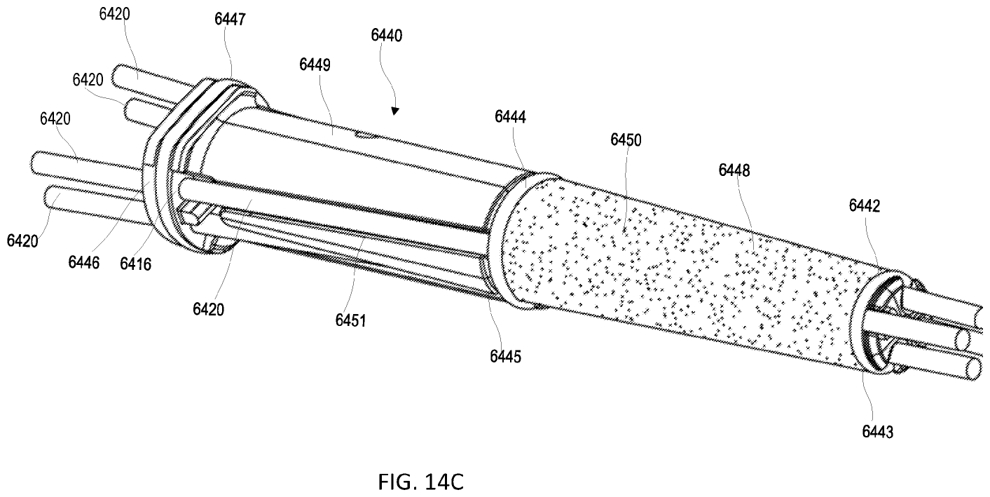

The tension element guide 6440 is coupled to a distal end portion of the shaft 6410 and is received within the shaft channel 6413. The tension element guide 6440 includes a proximal seal 6442 at a proximal location 6443, a middle seal 6444 at a middle location 6445, and a distal seal 6446 at a distal location 6447. The proximal seal 6442 and the middle seal 6444 are each an O-ring positioned on an outer surface 6441 of the tension element guide 6440. In some embodiments, the O-rings can be formed with a silicone material. The tension element guide 6440 also defines a sealing substance volume 6448 between the proximal seal 6442 and the middle seal 6424 and between an inner surface 6915 of the spacer 6900 and the outer surface 6441 of tension element guide 6440. As shown in FIG. 14A, the sealing substance volume 6448 can be defined, at least in part, by one or more grooves within outer surface 6441 of the tension element guide 6440. A sealing substance 6450 (see FIG. 14C) is within the sealing substance volume 6448. The sealing substance 6450 provides a seal to prevent fluid (e.g., gas and/or liquid) from traveling from a location distal of the middle seal 6444 to a location proximal of the proximal seal 6442. For example, during use of the medical device 6400, electrically conductive liquid (e.g., body fluid, surgical irrigation water) is susceptible to being drawn via capillary action (e.g., wicking) from outside the distal end of the medical device 6400 via the tension element 6420 to inside the shaft 6410. In some situations, fluid can enter the medical device via insufflation pressure. The sealing substance 6450 prevents such capillary action and prevents fluids from reaching the electrically conductive shaft 6410 (e.g., formed with a material such as stainless steel) and causing unintended damage to tissue.

The sealing substance 6450 can be, for example, a human biocompatible semi-solid sealing substance such as, grease, lubricant, gel, etc. In some embodiments, the sealing substance is NuSil™ MED-422 Silicone Fluid. In some embodiments, the sealing substance 6450 is a hydrophobic substance.

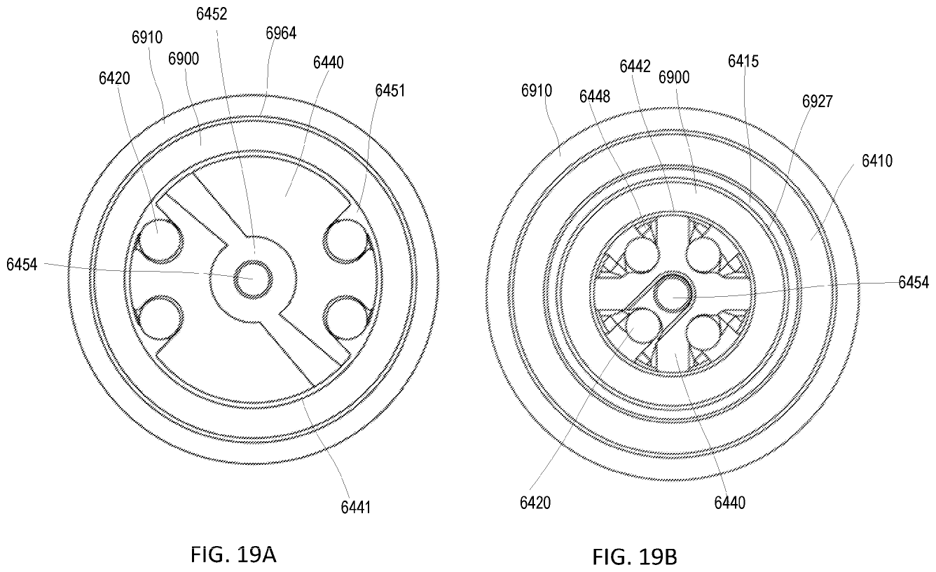

The tension element guide 6440 also includes an intermediate portion 6449 between the middle seal 6444 and the distal seal 6446 through which the tension elements 6420 extend. The intermediate portion 6449 defines guide passageways 6451 that extend to corresponding passageways within the sealing substance volume 6448 as shown, for example, in FIGS. 14A-14C. See also, FIG. 19B which illustrates the passageways within the sealing substance volume 6448 within which the tension elements 6420 extend. More specifically, the tension elements 6420 extend distally from the shaft channel 6413 of the shaft 6410, past the proximal seal 6442, through the sealing substance 6450 in the guide passageways of the sealing substance volume 6448, past the middle seal 6444, within the guide passageways 6451 of the intermediate portion 6449, past the distal seal 6446, and distally beyond the tension element guide 6440. The tension elements 6420 shown in FIGS. 13, 14B, 14C, 16 and 17 show the tension elements 6420 ending distally of the tension element guide 6440 for illustration purposes only. As described herein, the tension elements 6420 can extend from the proximal mechanical structure to the end effector 6460. Further, four sections of tension elements 6420 are shown, which can include one or more tension elements 6420. For example, a single tension element 6420 can extend from the proximal mechanical structure to the wrist assembly, and back to the proximal mechanical structure, showing two sections in the figures (see, e.g., FIG. 5A).

As shown, the tension elements 6420 pass under the proximal seal 6442 and the middle seal 6444 and through tension element openings 6416 of the distal seal 6446 described in more detail below. Said another way, the tension elements 6420 are routed between the outer surface of the tension element guide 6440 and each of the proximal seal 6442 and the middle seal 6444, and through the tension element openings 6416. In some embodiments, the tension element 6420 is a braided polymeric cable as described above that includes multiple individual strands. With the tension element 6420 passing through the sealing substance 6450, the sealing substance 6450 becomes deposited between the individual strands of the tension element 6450 such that a capillary action (e.g., wicking) is prevented between the individual strands of the tension element 6420. This prevents fluids from traveling in a proximal direction along the tension element 6420 from a location distal of the tension element guide 6440 to a location proximal of the proximal seal 6442.

As described above, the end effector 6460 can have a defined full range of motion as described herein for other embodiments. The tension elements 6420 are coupled to the end effector 6460 and translates a preset distance within the shaft 6410 and the sealing substance volume 6448 (e.g., proximally and distally) to move the end effector 6460 through the full range of motion of the end effector 6460. Similarly stated, the medical device 6400 is configured such that the tension elements 6420 has a travel distance that corresponds to movement of the end effector 6460 through its full range of motion. The distal seal 6446 is spaced apart from the middle seal 6444 by a distance D (see FIG. 18A) that is larger than the preset distance that the tension element 6420 travels. The spacing between the distal seal 6446 and the middle seal 6444 prevents sealing substance 6450 deposited on the tension element 6420 from being transported distally beyond the distal seal 6446. This arrangement limits the likelihood that the sealing substance 6450 will contact the end effector 6460.

The distal seal 6446 is overmolded to the tension element guide 6440. The distal seal 6446 can be formed with, for example, Santoprene™ thermoplastic vulcanizates (TPVs), a thermoplastic polyurethane (TPU) material, or other suitable material. The distal seal 6446 includes the tension element openings 6416, through which the tension elements 6420 pass through (see, e.g., FIGS. 13, 14B, 14C, and 15). The tension element openings 6416 can be shaped and sized to also limit fluids (e.g., liquids and/or insufflation gas) from leaking through the openings 6416 while also allowing the tension elements 6420 to move within the openings 6416. For example, the tension element openings 6416 can be shaped and sized as described and shown for tension element openings 2416 and have an hourglass shape that has a first tapered portion at a first end of the openings 6416, a second tapered portion at a second end, and a throat portion. The hourglass shape of the tension element openings 6416 and the diameter of the openings 6416 provide for a tight fit to the tension element 6420 when the tension element is under tension to prevent liquids and/or gas leakage through the openings 6416.

The tension element guide 6440 also includes an electrical conductor seal 6452 within an interior of the tension element guide 6440. In this embodiment, the electrical conductor seal 6452 is between the middle seal 6444 and the distal seal 6446, as shown in FIGS. 18A and 18B. The electrical conductor seal 6452 can be formed with, for example, Santoprene™ thermoplastic vulcanizates (TPVs), a thermoplastic polyurethane (TPU) material, or other suitable material. The electrical conductor seal 6452 is overmolded to the tension element guide 6440. For example, as shown in FIG. 19A, the electrical conductor seal 6452 extends from a center portion of the tension element guide 6440 to the outer surface 6441 of the tension element guide 6440 where the material for the electrical conductor seal 6452 is injected. The electrical conductor 6454 extends through the shaft 6410, past the proximal seal 6442, past the middle seal 6444, and through the electrical conductor seal 6452 and connects to the proximal link 6511 as described above. The electrical conductor seal 6452 prevents fluids from traveling from a location distal of the electrical conductor seal 6452 to a location proximal of the electrical conductor seal 6452.

In some embodiments, the tension element guide 6440 can be electrically insulative and can be formed with an insulative material, such as, for example, a polyphthalamide PPA GF material, or other suitable material. The tension element guide 6440 can be a single monolithically constructed component or can include multiple components coupled together. In some embodiments, the tension element guide 6440 includes a first sealing substance volume (e.g., 6448) and a second sealing substance volume (not shown) and the sealing substance 6450 is within both the first and second sealing substance volumes. In some embodiments, the first and second sealing substance volumes are interconnected. In other words, the sealing substance 6450 can move between the first and second sealing substance volumes. In some embodiments, the first and second sealing substance volumes are not interconnected (i.e., the first and second sealing substance volumes are closed from each other). In such embodiments, separate tension elements 6420 or sections of tension elements 6420 can pass through different separated sealing substance volumes.

The spacer 6900 can be electrically insulative and formed with, for example, a polyphthalamide PPA GF material, or other suitable material. The spacer 6900 includes the inner surface 6915 that defines an interior passageway 6946 (see FIGS. 12B and 16-18B) and the tension element guide 6440 is positioned within the interior passageway 6946. The spacer 6900 includes the flanges 6931 and openings 6930 through which the spring pin 6525 is received to couple the proximal link 6510 to the spacer 6900 as described above. The spacer 6900 also includes a proximal coupling portion 6917 that includes coupling ribs 6927. The proximal coupling portion 6917 is received within a distal end of the inner insulative cover 6415 and the coupling ribs 6927 engage an inner surface of the inner insulative cover 6415.

The spacer 6900 also provides sealing surfaces to prevent fluid from outside a distal end of the medical device 6400 from passing within the medical device 6400 and reaching the shaft 6410. More specifically, as shown in FIGS. 18A and 18B, the spacer 6900 includes a sealing protrusion 6913 that engages an inner surface of the distal tip cover 6940 described in more detail below. The spacer 6900 further includes a sealing protrusion 6964 and a sealing protrusion 6966 that both engage an inner surface 6939 of the outer insulative cover 6910 and provide a seal between the spacer 6900 and an inner surface 6939 of the outer insulative cover 6910 to prevent fluid from passing therebetween. The spacer can also optionally include additional sealing surfaces similar to or the same as the sealing surfaces shown and described in co-pending U.S. Provisional Patent Application Ser. No. 63/294,103, entitled “Surgical Instrument Including Electrical and Fluid Isolation Features,” filed Dec. 28, 2021, incorporated by reference herein in its entirety.

As shown in FIGS. 18A and 18B, the distal tip cover 6940 is coupled to the spacer 6900 and is positioned over the wrist assembly 6500 and a portion of the end effector 6460. The distal tip cover 6940 can be formed with, for example, silicone or other suitable materials. The distal tip cover 6940 includes an inner tip cover 6945 that is coupled to an interior surface of the distal tip cover 6940 and can be formed with, for example, TPU or other suitable material. The distal tip cover 6940 can protect tissue in the treatment area from electrically charged wrist assembly components, and the inner tip cover 6945 can protect the distal tip cover 6940 from damage caused by movement of the cutting shears. The inner tip cover 6945 includes a coupling protrusion 6942 that is positioned within a mounting recess 6936 of the spacer 6900 between the outer cover 6910 and the spacer 6900 and contacts the sealing protrusion 6913 with an interference fit such that the inner tip cover 6945 deforms at the sealing protrusion 6913 as shown in FIGS. 18A and 18B. The distal tip cover 6940 and the outer insulative cover 6910 prevent or limit the shaft 6410 from being exposed to liquids and/or insufflation gases during use of the medical device 6400 during a procedure. The distal tip cover 6940 and the outer cover 6910 also isolate the electrically conductive shaft 6410 such that if the shaft 6410 becomes capacitively coupled and charged during a procedure, the shaft 6410 will not cause unintended damage to tissue.

As described herein, the medical device 6400 can provide electrical energy to the end effector 6460 to electrically charge the tool members 6462 and 6482 to cut or cauterize tissue. The wrist assembly 6500 may include components formed in part with electrically conductive material to deliver energy to the tool. For example, the first link 6510 and the second link 6610 can be formed with a metallic material. As described above, the proximal portion 6511 of the link 6510 includes an interior region 6528 that can receive and be coupled to an electrical conductor 6454. As described herein, during a procedure using the medical device 6400, it is desirable to prevent or limit fluid contact with the electrically conductive shaft 6410 which acts as an energy shield. The shaft 6410 can become capacitively coupled and charged during the application of energy and to avoid unintended damage to tissue during treatment using the medical device 6400, the distal tip cover 6940, outer cover 6910, spacer 6900, tension element guide 6440, sealing substance 6450, and inner insulative cover 6415 are provided to insulate the shaft 6410 from electrical energy.

In some embodiments, the medical device 6400 also includes an energy shield monitoring system (ESM) (not shown) that can detect if there has been a breach that can cause electrical contact between anatomical tissue and the shaft 6410. For example, the ESM system can detect if there is a breach in the outer cover 6910, inner cover 6415, and/or spacer 6900. If detected, the ESM system can be triggered to turn off the electrical power to the wrist assembly 6500. In some embodiments, the ESM system can be configured the same as or similar to and function the same as or similar to, the systems described in International Application No. PCT/U S2018/035436 (Publication No. WO 2018/222899), the disclosure of which is incorporated herein by reference, or in U.S. Provisional Patent Application Ser. No. 63/294,103 incorporated by reference above.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument described herein can be constructed from any material, such as medical grade stainless steel, nickel alloys, titanium alloys or the like. Further, any of the links, tool members, tension elements, or components described herein can be constructed from multiple pieces that are later joined together. For example, in some embodiments, a link can be constructed by joining together separately constructed components. In other embodiments however, any of the links, tool members, tension elements, or components described herein can be monolithically constructed.

Although the instruments are generally shown as having an axis of rotation of the tool members (e.g., axis A1) that is normal to an axis of rotation of the wrist member (e.g., axis A2), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device, comprising:
an instrument shaft including a proximal end portion, a distal end portion, and a channel between the proximal end portion and the distal end portion;
a tension element guide at the distal end of the instrument shaft, the tension element guide including a proximal seal, a distal seal, and a middle seal between the proximal seal and the distal seal;
a tension element; and
a semisolid sealing substance;
wherein a sealing substance volume is defined between the proximal seal and the middle seal;
wherein the sealing substance volume holds the sealing substance; and
wherein the tension element extends distally from the channel of the instrument shaft, past the proximal seal, through the sealing substance in the sealing substance volume, past the middle seal, past the distal seal, and distally beyond the tension element guide.

2. The medical device of claim 1, wherein:
the medical device includes a spacer at the distal end portion of the shaft;
the spacer includes an inner surface;
the tension element guide includes an outer surface; and
the sealing substance volume is defined between the inner surface of the spacer and the outer surface of the tension element guide.

3. The medical device of claim 2, wherein:
the spacer and the tension element guide are each electrical insulators.

4. The medical device of claim 1, wherein:
the middle seal includes an O-ring that extends around the tension element guide; and
the tension element passes under the O-ring.

5. The medical device of claim 1, wherein:
the proximal seal includes an O-ring that extends around the tension element guide; and
the tension element passes under the O-ring.

6. The medical device of claim 1, wherein:
the medical device includes a movable component coupled to the distal end portion of the shaft;
the movable component has a defined full range of motion;
the tension element is coupled to the movable component and translates a first distance to move the movable component through the full range of motion of the movable component; and
the distal seal is spaced apart from the middle seal by a second distance larger than the first distance.

7. The medical device of claim 1, wherein:
the tension element is a braided polymeric cable that includes a plurality of individual strands; and
within the sealing volume, the sealing substance is between the individual strands of the plurality of individual strands such that a capillary action is prevented between the individual strands of the plurality of individual strands.

8. The medical device of claim 1, wherein:
the tension element is a first tension element, and the sealing substance volume is a first sealing substance volume;
the medical device includes a second tension element;
a second sealing substance volume is defined between the proximal seal and the middle seal;
the second sealing substance volume holds a portion of the sealing substance; and
the second tension element extends from the channel of the instrument shaft, past the proximal seal, through the sealing substance in the second sealing substance volume, past the middle seal, past the distal seal, and distally beyond the tension element guide.

9. The medical device of claim 8, wherein:
the first sealing substance volume and the second sealing substance volume are interconnected within the tension element guide.

10. The medical device of claim 1, wherein:
the sealing substance includes a hydrophobic substance.

11. The medical device of claim 1, wherein:
the tension element guide is a single monolithic piece.

12. The medical device of claim 1, wherein:
the distal seal is overmolded to the tension element guide.

13. The medical device of claim 1, wherein:
the medical device includes an electrical conductor seal; and
an electrical conductor that extends through the electrical conductor seal.

14. The medical device of claim 13, wherein:
the electrical conductor seal is overmolded to the tension element guide.

15. The medical device of claim 1, wherein:
the instrument shaft is electrically conductive.

16. A medical device, comprising:
an instrument shaft including a proximal end portion, a distal end portion, and a channel between the proximal end portion and the distal end portion;
a tension element guide at the distal end of the instrument shaft, the tension element guide including a proximal location, a distal location, and a middle location between the proximal location and the distal location;
a tension element including a plurality of strands; and
a semisolid sealing substance;
wherein a sealing substance volume is defined between the proximal location of the tension element guide and the middle location of the tension element guide;
wherein the sealing substance volume holds the sealing substance;
wherein the tension element extends distally from the channel of the instrument shaft, past the proximal location of the tension element guide, through the sealing substance in the sealing substance volume, past the middle location of the tension element guide, and beyond the distal location of the tension element guide;
wherein the tension element is movable proximally and distally within the sealing substance volume; and
wherein fluid is prevented from traveling in a proximal direction along the tension element from a location distal of the distal location of the tension element guide to a location proximal of the proximal location of the tension element guide by sealing substance from the sealing substance volume being between individual strands of the plurality of strands of the tension element.

17. The medical device of claim 16, wherein:
the medical device includes a proximal seal at the proximal location of the tension element guide, a middle seal at the middle location of the tension element guide, and a distal seal at the distal location of the tension element guide; and
the middle seal and the distal seal together prevent the sealing substance from migrating from within the sealing substance volume to a location distal of the distal seal.

18. The medical device of claim 17, wherein:
the middle seal includes an O-ring that extends around the tension element guide; and
the tension element passes under the O-ring.

19. The medical device of claim 17, wherein:
the proximal seal includes an O-ring that extends around the tension element guide; and
the tension element passes under the O-ring.

20. The medical device of claim 17, wherein:
the distal seal includes a tension element opening;
the tension element opening includes a first taper portion, a second taper portion, and a throat;
the throat defines the diameter of the tension element opening;
the tension element has a first diameter when the tension element is in an untensioned state and a second diameter when the tension element is in a tensioned state;
the first diameter of the tension element is larger than the diameter of the tension element opening; and
the second diameter of the tension element is less than the diameter of the tension element opening.

21. The medical device of claim 17, wherein:
the medical device includes a movable component coupled to the distal end portion of the shaft;
the movable component has a defined full range of motion;
the tension element is coupled to the movable component and translates a first distance to move the movable component through the full range of motion of the movable component; and
the distal seal is spaced apart from the middle seal by a second distance larger than the first distance.

22. The medical device of claim 16, wherein:
the medical device includes a spacer at the distal end portion of the shaft;
the spacer includes an inner surface;
the tension element guide includes an outer surface; and
the sealing substance volume is defined between the inner surface of the spacer and the outer surface of the tension element guide.

23. The medical device of claim 22, wherein:
the spacer and the tension element guide are each electrical insulators.

24. The medical device of claim 17, wherein:
the distal seal is overmolded to the tension element guide.

25. The medical device of claim 16, wherein:
the medical device includes an electrical conductor seal; and
an electrical conductor that extends through the electrical conductor seal.

26. The medical device of claim 16, wherein:
the instrument shaft is electrically conductive.
